(19) United States
(12) Patent Application Publication
(10) Pub. No.: US 2026/0274858 A1
(43) Pub. Date: Sep. 17, 2026

(54) POLYIMIDE, ALICYCLIC DIANHYDRIDE CONTAINING ETHER GROUP, AND SYNTHESIS METHOD THEREOF

(71) Applicant: National Taiwan University of Science and Technology, TAIPEI CITY (TW)

(72) Inventors: Jyh-Chien CHEN, Hsinchu City (TW); Yueh-Han LI, Taipei City (TW); Shih-Jhong WU, New Taipei City (TW); Kai-Cheng ZHONG, Kaohsiung City (TW); Wen-Xiu LI, Tainan City (TW); Hsiang Jung LEE, Taipei City (TW)

(21) Appl. No.: 19/242,963

(22) Filed: Jun. 18, 2025

(30) Foreign Application Priority Data
Mar. 12, 2025 (TW) 114109023

(51) Int. Cl. C07D 493/08

(52) U.S. Cl. CPC C07D 493/08

(57) ABSTRACT

The present disclosure provides a polyimide, an alicyclic dianhydride containing ether group, and synthesis method thereof. The alicyclic dianhydride containing ether group has a structure of the following formula (I), formula (II), or formula (III):

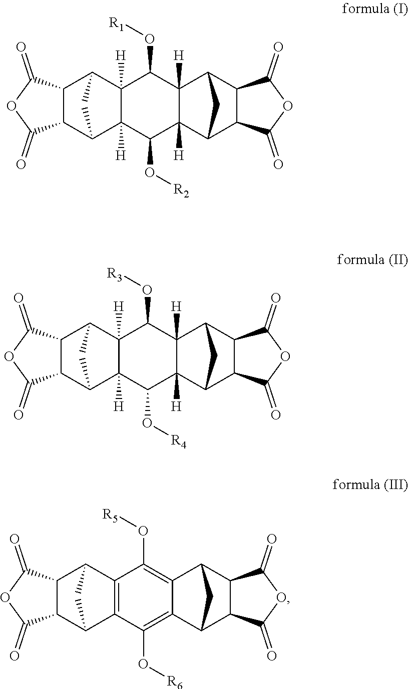

formula (III), wherein R₁, R₂, R₃, R₄, R₅, R₆ independently comprise a C₁ to C₅ hydrocarbon group.

### CROSS-REFERENCE TO RELATED APPLICATION

This application claims priority to Taiwan Application Serial Number 114109023, filed Mar. 12, 2025, which is herein incorporated by reference in its entirety.

### BACKGROUND

### Field of Invention

The present disclosure relates to a polyimide, an alicyclic dianhydride containing ether group, and a synthesis method of an alicyclic dianhydride containing ether group.

### Description of Related Art

Polyimide (PI) is a polymer with excellent thermal and mechanical properties as it is widely used in the industry nowadays. PI has two variants derivatizing from aromatic and aliphatic chemical structures. Particularly to the optoelectronic industry, conventional aromatic polyimides normally have color which cannot be introduced of making transparent optical films. Aliphatic polyimide otherwise is more suitable for producing transparent optical films with its transparency and lower charge transfer interaction on such use. However, synthesis process of aliphatic monomers for manufacturing aliphatic polyimides has problem of isomers and other by-products which become more difficult to separate and purify for determine the actual structure of synthesized compounds. These uncontrollable factors result in the limitation of use for aliphatic polyimides. Therefore, a refine synthesis path of such monomer has become one of the most critical point in the development of aliphatic polyimides.

In view of the above, it is eager to have a solution that will overcome or substantially ameliorate at least one or more of the deficiencies of a prior art, or to at least provide an alternative solution to the problems.

### SUMMARY

In order to solve the problem that the traditional aromatic polyimide cannot be used in transparent optical films due to its color, and even if it is replaced with alicyclic polyimide, it is still difficult to separate and purify the alicyclic monomer and to determine its configuration, the present disclosure develops a novel ether-based alicyclic dianhydride monomer using a completely new synthesis route. And the monomer was used to successfully synthesize polyimide with excellent thermal stability, solubility, processability and colorless and transparent film.

The present disclosure provides an alicyclic dianhydride containing ether group represented by a structure of the following formula (I), formula (II), or formula (III):

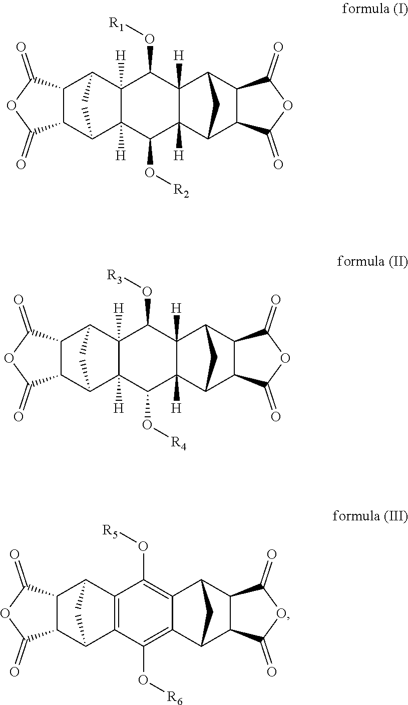

formula (III), wherein R₁, R₂, R₃, R₄, R₅, and R₆ independently include a C1 to C5 hydrocarbon group.

The present disclosure further provides a synthesis method of an alicyclic dianhydride containing ether group, which includes the following steps. A methoxy carbonylation reaction of a compound 1a is performed to obtain a compound 1b, and a hydrolysis and dehydration reaction of the compound 1b is performed to obtain the alicyclic dianhydride containing ether group represented by a formula (I),

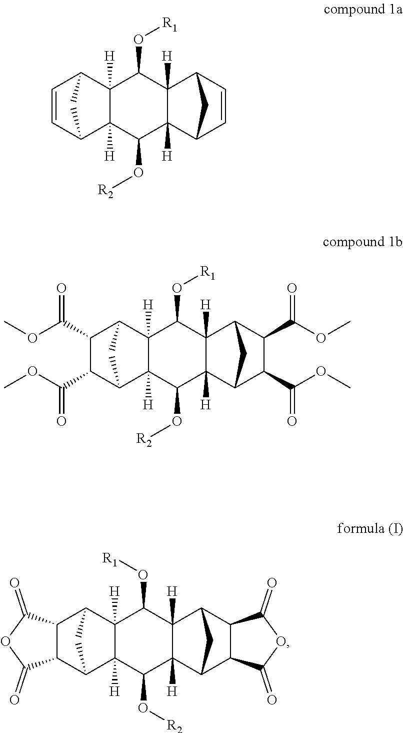

wherein R₁, R₂ independently include a C1 to C5 hydrocarbon group. Alternatively, a methoxy carbonylation reaction of a compound 2a is performed to obtain compound 2b, and a hydrolysis and dehydration reaction of the compound 2b is performed to obtain the alicyclic dianhydride containing ether group represented by a formula (II),

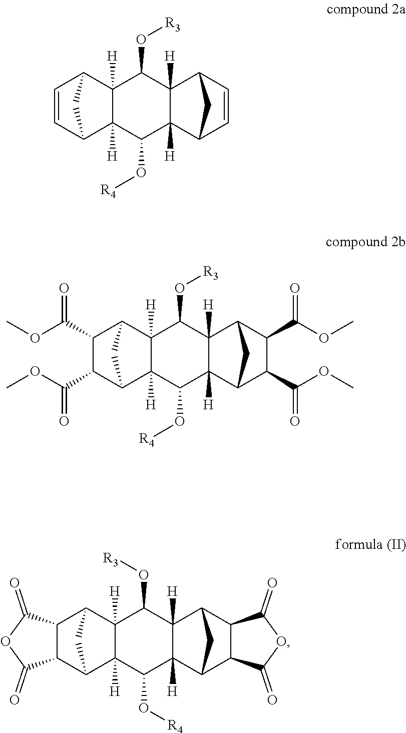

wherein R₃, R₄ independently include a C1 to C5 hydrocarbon group. Another option is performing a methoxy carbonylation reaction of a compound 3a to obtain compound 3b, and performing a hydrolysis and dehydration reaction of the compound 3b to obtain the alicyclic dianhydride containing ether group represented by a formula (III),

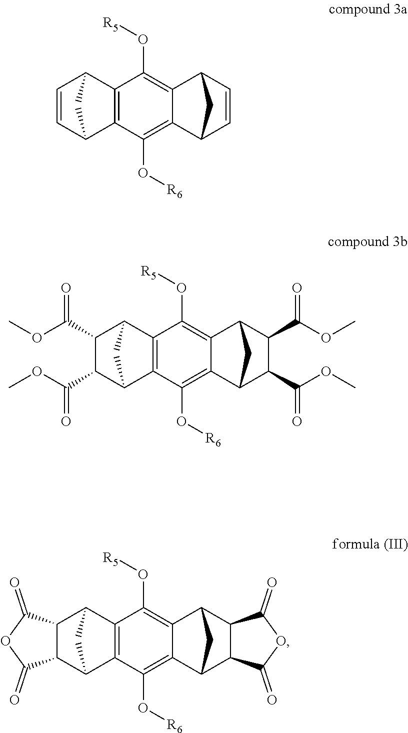

wherein R₅, R₆ independently include a C1 to C5 hydrocarbon group.

The present disclosure further provides a polyimide represented by a structure of the following formula (IV), formula (V), formula (VI), formula (VII), formula (VIII), or formula (IX):

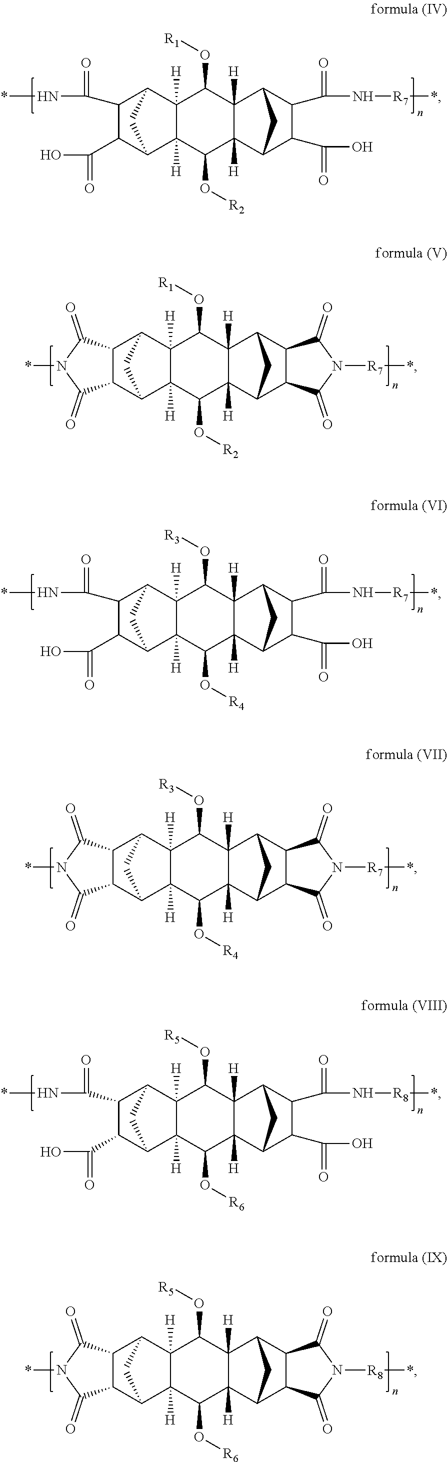

wherein R₁, R₂, R₃, R₄, R₅, and R₆ independently include a C1 to C5 hydrocarbon group, R₇ and R₈ independently include an aromatic group, an aliphatic group, a combination of an aromatic group and

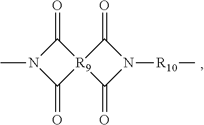

or a combination of aliphatic group and

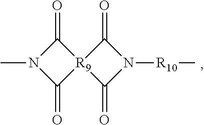

wherein R₉ is a moiety independently selected from the group consisting of substituted or unsubstituted aromatic, heterocyclic aromatic and aliphatic groups, Rio comprises an aromatic group or an aliphatic group, and n is a positive integer of 1 to 500.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram of the synthesis process of an alicyclic dianhydride monomer containing ether group according to the present disclosure.

FIG. 2A, FIG. 2B, FIG. 2C, FIG. 2D and FIG. 2E are ¹H NMR spectrum, ¹H¹H—COSY spectrum, ¹³C NMR spectrum, ¹³C¹H—HSQC spectrum, and X-ray single crystal diffraction spectrum of compound 1-1 in FIG. 1, respectively.

FIG. 3A, FIG. 3B, FIG. 3C, FIG. 3D and FIG. 3E are ¹H NMR spectrum, ¹H¹H—COSY spectrum, ¹³C NMR spectrum, ¹³C¹H—HSQC spectrum, and X-ray single crystal diffraction spectrum of compound 1-2 in FIG. 1, respectively.

FIG. 4A, FIG. 4B, FIG. 4C, FIG. 4D and FIG. 4E are ¹H NMR spectrum, ¹³C NMR spectrum, ¹³C¹H—HSQC spectrum, EI-MASS spectrum, and X-ray single crystal diffraction spectrum of compound 2 in FIG. 1, respectively.

FIG. 5 is a Thermogravimetric analysis (TGA) spectrum of a polyimide derived from an alicyclic dianhydride containing ether group according to various embodiments of the present disclosure.

FIG. 6 is a UV-visible spectrum of a polyimide film derived from an alicyclic dianhydride containing ether group according to various embodiments of the present disclosure.

FIG. 7 is a Fourier transform infrared spectrum of a polyimide film derived from an alicyclic dianhydride containing ether group according to various embodiments of the present disclosure.

### DETAILED DESCRIPTION

To facilitate of understanding and reading the present invention, all the following process of synthesis of this novel alicyclic dianhydride containing ether group will be described in sequence. However, it should be understandable yet more reasonable that the process of synthesis provided below is only preferable embodiments. Other reasonable adjustments in the same or similar process or steps shall be covered by the present invention.

The present disclosure provides an alicyclic dianhydride containing ether group and a synthesis method thereof. To be specific, the alicyclic dianhydride containing ether group represented by a structure of the following formula (I), formula (II), or formula (III):

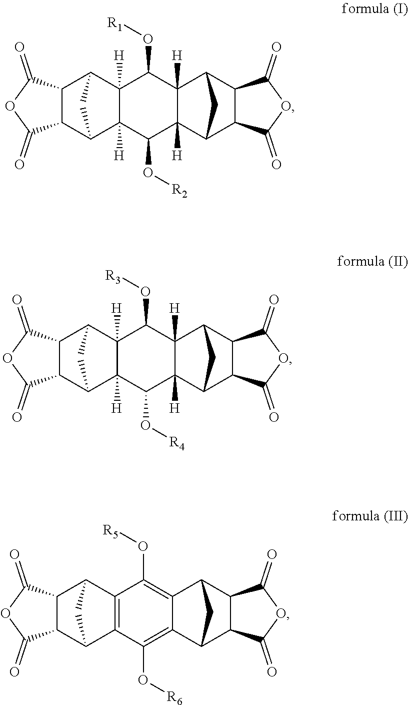

wherein R₁, R₂, R₃, R₄, R₅, and R₆ independently include a C1 to C5 hydrocarbon group.

More specifically, the synthesis method of the alicyclic dianhydride containing ether group represented by the formula (I) includes performing a methoxy carbonylation reaction of a compound 1a to obtain a compound 1b, and performing a hydrolysis and dehydration reaction of the compound 1b to obtain the alicyclic dianhydride containing ether group represented by the formula (I),

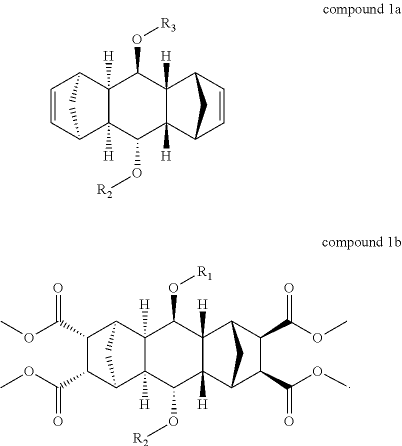

In some embodiments, the compound 1a is obtained by performing a Williamson ether synthesis reaction of the compound 1c. The Williamson ether synthesis reaction includes adding a haloalkane. The haloalkane includes a C1 to C5 haloalkane. The compound 1c is represented by a structure as shown below:

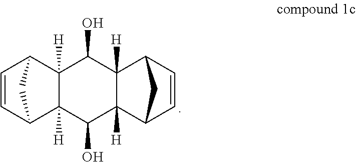 [SMILES: O[C@H]1[C@@H]2[C@@H]3C=C[C@@H](C3)[C@@H]2[C@@H](O)[C@H]2[C@H]3C=C[C@H](C3)[C@@H]12]

For example, the alicyclic dianhydride containing ether group is represented by the formula (I) may be

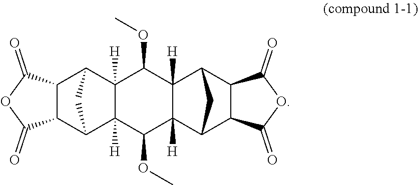 [SMILES: CO[C@H]1[C@@H]2[C@H]3C[C@H]([C@@H]4C(=O)OC(=O)[C@@H]43)[C@@H]2[C@@H](OC)[C@H]2[C@@H]3C[C@@H]([C@H]4C(=O)OC(=O)[C@H]43)[C@@H]12]

For another example, the alicyclic dianhydride containing ether group is represented by the formula (I) may be

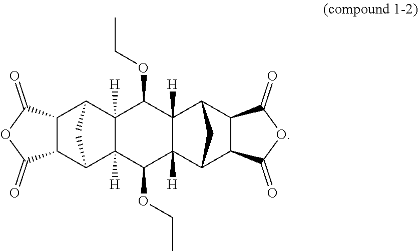 [SMILES: CCO[C@H]1[C@@H]2[C@H]3C[C@H]([C@@H]4C(=O)OC(=O)[C@@H]43)[C@@H]2[C@@H](OCC)[C@H]2[C@@H]3C[C@@H]([C@H]4C(=O)OC(=O)[C@H]43)[C@@H]12]

More specifically, the synthesis method of the alicyclic dianhydride containing ether group represented by the formula (II) includes performing a methoxy carbonylation reaction of a compound 2a to obtain compound 2b, and performing a hydrolysis and dehydration reaction of the compound 2b to obtain the alicyclic dianhydride containing ether group represented by the formula (II), of which the compound 2a is represented by the structure shown below:

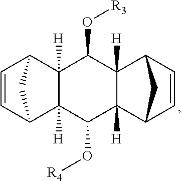

and the compound 2b is represented by the structure shown below:

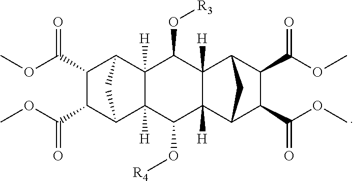

In some embodiments, the compound 2a is obtained by performing a Williamson ether synthesis reaction of the compound 2c. The Williamson ether synthesis reaction includes adding a haloalkane. The haloalkane includes a C1 to C5 haloalkane.

For example, the alicyclic dianhydride containing ether group is represented by the formula (II) may be

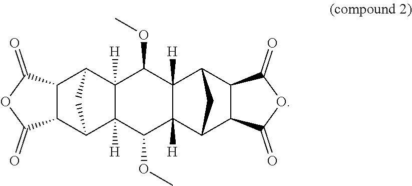 [SMILES: CO[C@H]1[C@@H]2[C@H]3C[C@H]([C@@H]4C(=O)OC(=O)[C@@H]43)[C@@H]2[C@H](OC)[C@H]2[C@@H]3C[C@@H]([C@H]4C(=O)OC(=O)[C@H]43)[C@@H]12]

More specifically, the synthesis method of the alicyclic dianhydride containing ether group represented by the formula (III) includes performing a methoxy carbonylation reaction of a compound 3a to obtain a compound 3b, and performing a hydrolysis and dehydration reaction of the compound 3b to obtain the alicyclic dianhydride containing ether group represented by a formula (III), of which the compound 3a is represented by the structure shown below:

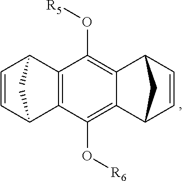

and the compound 3b is represented by the structure shown below:

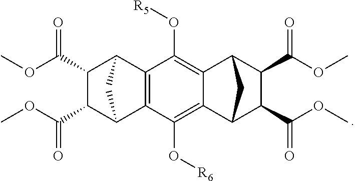

In some embodiments, the compound 3a is obtained by performing an oxidation and alkylation reactions of the compound 3c. The alkylation reaction includes adding a haloalkane. The haloalkane comprises a C1 to C5 haloalkane. The compound 3c is represented by a structure as shown below:

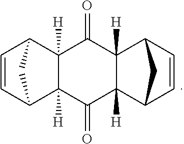 [SMILES: O=C1[C@@H]2[C@H](C(=O)[C@H]3[C@@H]1[C@H]1C=C[C@@H]3C1)[C@@H]1C=C[C@H]2C1]

For example, the alicyclic dianhydride containing ether group is represented by the formula (III) may be

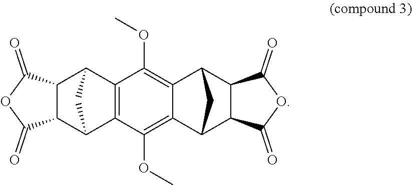 [SMILES: COc1c2c(c(OC)c3c1[C@H]1C[C@@H]3[C@@H]3C(=O)OC(=O)[C@@H]31)[C@@H]1C[C@H]2[C@H]2C(=O)OC(=O)[C@H]21]

The present disclosure further provides a polyimide polymer. To be specific, the present disclosure provides a polyimide polymer derived from the above-mentioned alicyclic dianhydride containing ether group. The polyimide represented by a structure of the following formula (IV), formula (V), formula (VI), formula (VII), formula (VIII), or formula (IX):

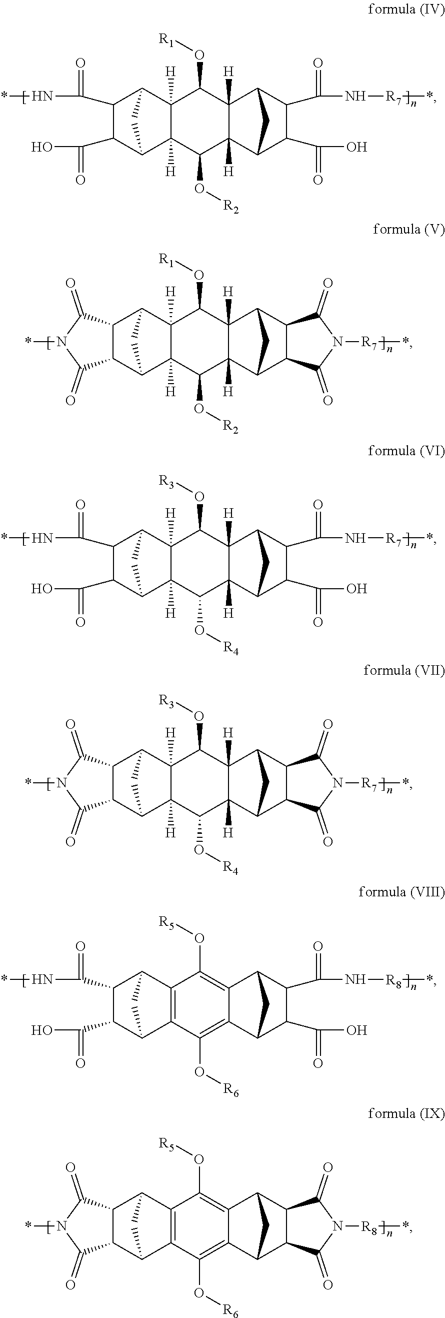

wherein R₁, R₂, R₃, R₄, R₅, and R₆ independently comprise a C1 to C5 hydrocarbon group, R₇ and R₈ independently include an aromatic group, an aliphatic group, a combination of an aromatic group and

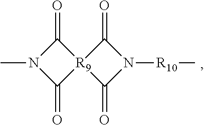

or a combination of aliphatic group and

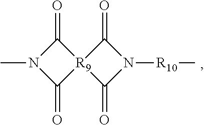

wherein R₉ is a moiety independently selected from the group consisting of substituted or unsubstituted aromatic, heterocyclic aromatic and aliphatic groups, Rio comprises an aromatic group or an aliphatic group, and n is a positive integer of 1 to 500.

The present disclosure is more particularly described in the following with reference to experiment examples. Although the following experiments are described, the material used, the material usage amount and ratio, processing details and processing procedures, etc., can be suitably modified without departing from the scope of the present disclosure. Accordingly, restrictive interpretation should not be made to the present disclosure based on the experiments described below.

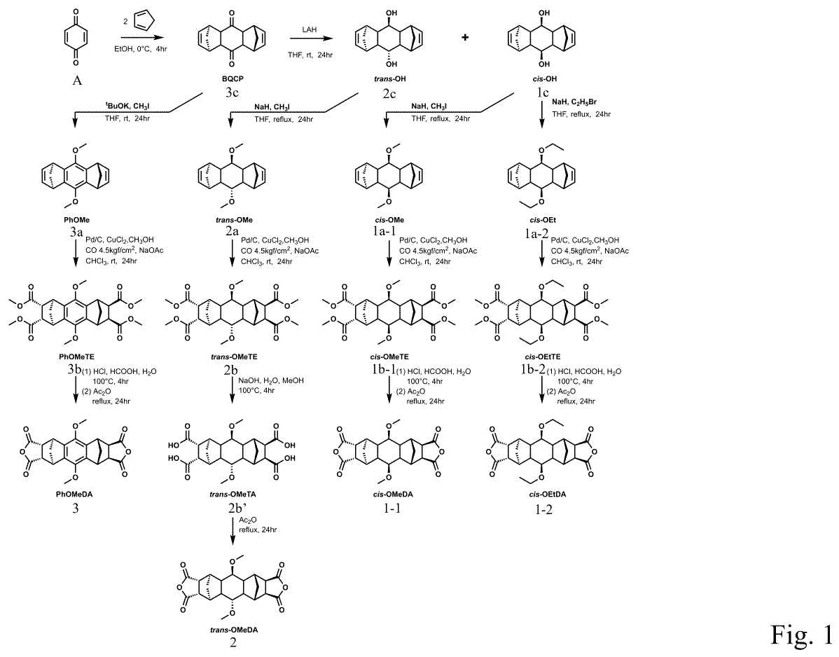

Please refer to the flowchart of FIG. 1, which discloses the preparation of the alicyclic dianhydride monomers containing ether group represented by formula (I), formula (II), and formula (III) and the structures of the intermediate compounds produced in each step. For example, the preferred embodiments of the present disclosure mainly provide a series of three alicyclic dianhydride monomers containing ether group, including alicyclic dianhydride monomers containing ether group of 1-1, 1-2, 2, and 3 as shown in FIG. 1, and having the stereoisomeric structural characteristics of the aforementioned compounds 1-1, 1-2, 2 and 3.

### Synthesis of Compound 1c (Cis-OH) and Compound 2c (Trans-OH):

The apparatus was set up with a dosing tube, a mechanical stirrer and a 500 mL three-necked flask flushed with nitrogen and dried using a spray gun to remove water. After cooling, 4.2 g (110.67 mmol) of lithium aluminium hydride (LAH) was added. Next, under an ice bath, 100 mL of dry tetrahydrofuran (dry THF) was drawn into the dosing tube using a double-ended needle and slowly dripped into the reaction bottle to form a suspension solution, and then the ice bath was removed. Since dry tetrahydrofuran still contains trace amounts of water, a large amount of heat is released when lithium aluminum hydride reacts with water. If the dry THE is not added slowly in an ice bath, some of the solvent will evaporate.

A 500 mL conical flask flushed with nitrogen was prepared and 8 g (33.29 mmol) of BQCP (compound 3c) was added to completely dissolve BQCP in 200 mL of dry tetrahydrofuran. The above solution was added into the dosing tube by using a double-ended needle. The stirring rate of the mechanical stirrer was increased, and 10 mL of the solution was slowly dripped into the three-necked flask at intervals of 30 seconds each time, so that the colloidal suspension solution was vigorously stirred and reacted for 12 hours.

After 12 hours, a saturated aqueous ammonium chloride solution (NH₄Cl_{(aq)}) was prepared to terminate the reaction. Specifically, 1 to 2 drops of saturated ammonium chloride aqueous solution were dripped into the three-necked flask every few minutes until no gas escapes after the dripping, and all the remaining lithium aluminum hydride in the three-necked flask was reacted with the ammonium chloride aqueous solution to form a white/gray suspension solution that was stirred for half an hour. This step must be done with extreme caution. If the ammonium chloride aqueous solution is added too quickly, the reaction will be so violent that the tetrahydrofuran will boil, and a large amount of hydrogen will be produced during the reaction.

The suspension was then poured into a beaker, supplemented with about 200 mL of tetrahydrofuran, and heated to 50° C. and stirred for 20 minutes. The suspended solution was filtered under vacuum while hot to collect the lower filtrate and then concentrated under reduced pressure to dry the solvent. The product was then washed with deionized water and n-hexane, and the upper solid was collected by vacuum filtration to obtain a white solid mixture of compound 1c (cis-OH) and compound 2c (trans-OH). The mixture could be filtered under vacuum with a rinse ratio of 1 g of solid to 50 mL of ethyl acetate. The white solid remaining on the filter cake is compound 1c (cis-OH), and after the ethyl acetate filtrate is drained, a white solid mixture of trace amounts of compound 1c (cis-OH) and compound 2c (trans-OH) is obtained. The filter cake on the upper layer after the first filtration was poured back into the beaker, about 500 mL of tetrahydrofuran was added, and the mixture was heated to 50° C. and stirred for 20 minutes. By repeating the above purification steps, almost pure compound 1c (cis-OH) was obtained as a white solid. If there is still a trace amount of compound 2c (trans-OH), it can be washed with ethyl acetate using the above method. The product is separated from those suspended solid impurities and dissolved in a solvent through two purification steps.

The solid mixture of compound 1c (cis-OH) and compound 2c (trans-OH) collected from the first and second ethyl acetate filtrates was recrystallized from ethyl acetate 2 to 3 times (1 g of compound 2c (trans-OH)/20 mL of ethyl acetate). Before repeated recrystallization, a glass funnel was used to filter out the precipitated fine white powder (the mixture with a relatively large proportion of compound 1c (cis-OH)), and the remaining majority of crystals was recrystallized once to obtain transparent crystals of compound 2c (trans-OH) with a purity of 99%.

### Synthesis of Compound 1a-1 (Cis-OMe):

A 1 L three-necked flask was flushed with nitrogen and equipped with a condenser and a magnetic stirrer. 6.90 g (173.12 mmol) of 60% sodium hydride and 5.30 g (21.69 mmol) of compound 1c (cis-OH) were added to the three-necked flask and dissolved in 550 mL of anhydrous tetrahydrofuran to form a white suspension liquid. After refluxing at 80° C. for 24 hours, about 50 mL of saturated aqueous ammonium chloride solution was added until the liquid was completely dissolved. The mixture was then extracted with saturated brine and tetrahydrofuran, and the organic layer was washed with saturated brine until it became neutral. The organic solution layer was collected. After removing the solvent by adding anhydrous magnesium sulfate and concentrating under reduced pressure, a yellow-brown oily paste product was obtained. After drying in a vacuum high-temperature oven at 100° C., 5.90 g (21.66 mmol) of compound 1a-1 (cis-OMe) was obtained with a yield of 99.9%.

### Synthesis of Compound 1b-1 (Cis-OMeTE):

In a 250 mL autoclave flushed with nitrogen and equipped with a magnetic stirrer, 5.90 g (21.66 mmol) of compound 1a-1 (cis-OMe) and 72 mL of anhydrous methanol were added. 20 mL of chloroform was added to make the liquids more miscible and form a mist and then 0.56 g (0.53 mmol) of 10% Pd/C and 19.87 g (147.79 mmol) of copper chloride were added in sequence, and the autoclave was locked. Then the carbon monoxide cylinder was connected to replace the nitrogen in the autoclave with carbon monoxide. After closing the air release valve, the pressure was adjusted to 4 kg/cm² and the mixture was stirred vigorously at room temperature for 1 day.

After the reaction is completed, the carbon monoxide in the autoclave is slowly released until the pressure returns to atmospheric pressure and no more gas is discharged. The autoclave was opened and 30 mL of chloroform was added into the reaction bottle and stirred for 15 minutes. The catalyst was removed by filtration through celite, and the filtrate was collected and extracted with saturated brine and chloroform. The lower organic layer was collected and then washed with saturated brine until it became neutral. After removing water by using anhydrous magnesium sulfate, a yellow-brown ointment could be obtained by removing chloroform using a vacuum concentrator. After adding a small amount of ether (about 5 mL) to the ointment and leaving it in the refrigerator overnight, yellow-brown crystals will precipitate at the bottom. After filtering, the product can be rinsed with ice ether to obtain white transparent crystals. The filtrate can be reacted again under the same conditions and dosage for 1 day. After extraction and precipitation by adding diethyl ether, white transparent crystals were obtained. A total of 4.50 g (8.85 mmol) of compound 1b-1 (cis-OMeTE) of white transparent crystals were obtained, with a total yield of 40.9%. If the obtained product still appears as slightly yellow crystals after being rinsed with ice ether, it can be purified by recrystallization using ethyl acetate to obtain white transparent crystals.

### Synthesis of Compound 1-1 (Cis-OMeDA):

The International Union of Pure and Applied Chemistry chemical nomenclature (IUPAC) of compound 1-1 is named as: (3aR,4R,4aR,5S,5aR,6R,6aR,9aS,10S,10aS,11R,11aS,12S,12aS)-5,11-dimethoxydodecahydro-4,12:6,10-dimethanoanthra[2,3-c:6,7-c′]difuran-1,3,7,9(3aH,9aH)-tetraone.

A 50 mL three-necked round-bottom flask flushed with nitrogen and equipped with a dosing tube, a condenser and a magnetic stirrer was added with 1.05 g (2.06 mmol) of compound 1b-1 (cis-OMeTE), 2 mL of formic acid, 2 mL of pure water and 0.4 mL of hydrochloric acid (37%) in sequence. The reaction temperature was adjusted to 90° C. and the mixture was completely dissolved after 4 hours of reaction. After the reaction was completed, the temperature was lowered to room temperature, and an off-white solid would slowly precipitate. 8 mL of acetic anhydride was slowly added by using a dosing tube to avoid sudden boiling. After the acetic anhydride is completely added, adjust the temperature to 120° C. After 12 hours of reaction, the temperature was lowered to room temperature, and then the condenser was replaced with a distillation tube and the dosing tube was removed. After complete evaporation of the solvent, a foamy yellow-white solid was obtained. A trace amount (about 2 mL) of acetic anhydride was added for recrystallization, and the product was placed in a vacuum oven at 200° C. for 12 hours to obtain 0.67 g (1.61 mmol) of compound 1-1 (cis-OMeDA) as white transparent crystals with a yield of 78.1%.

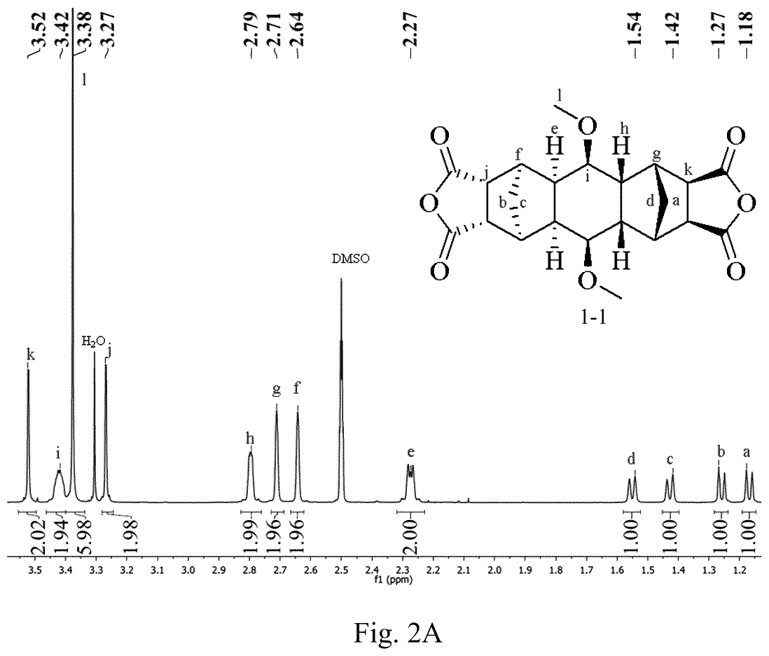

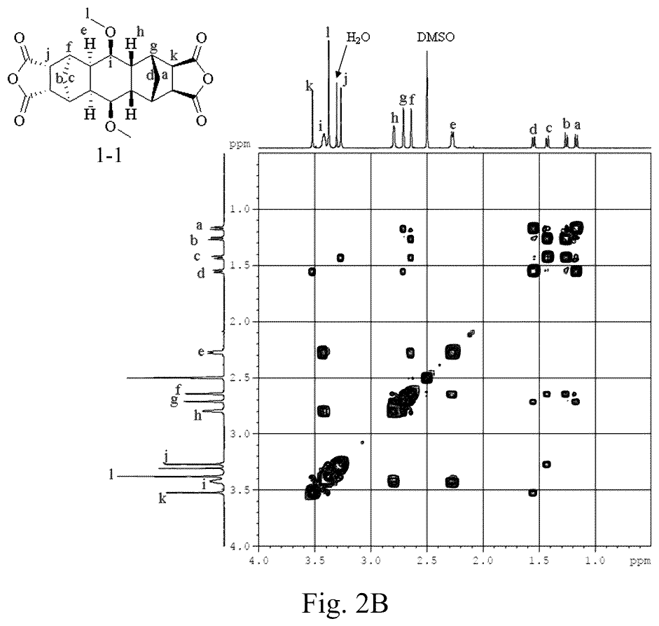

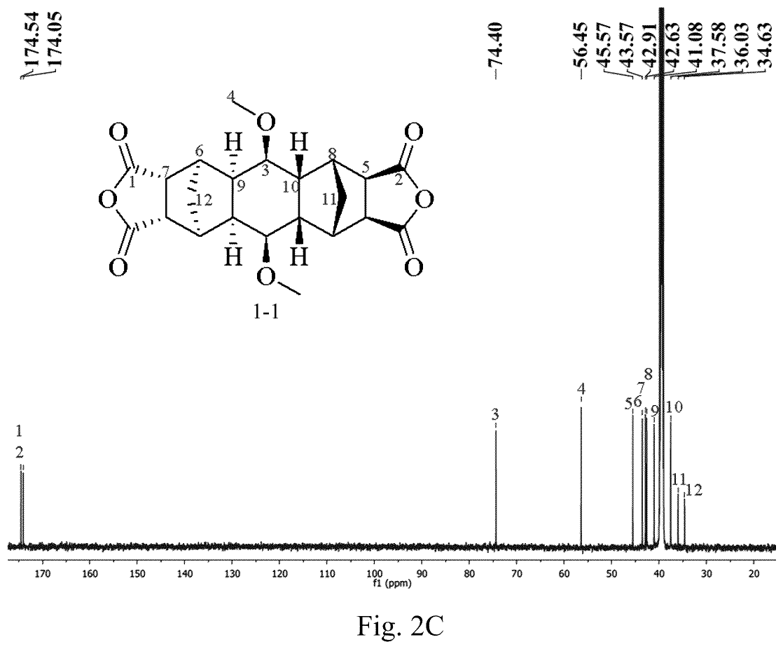

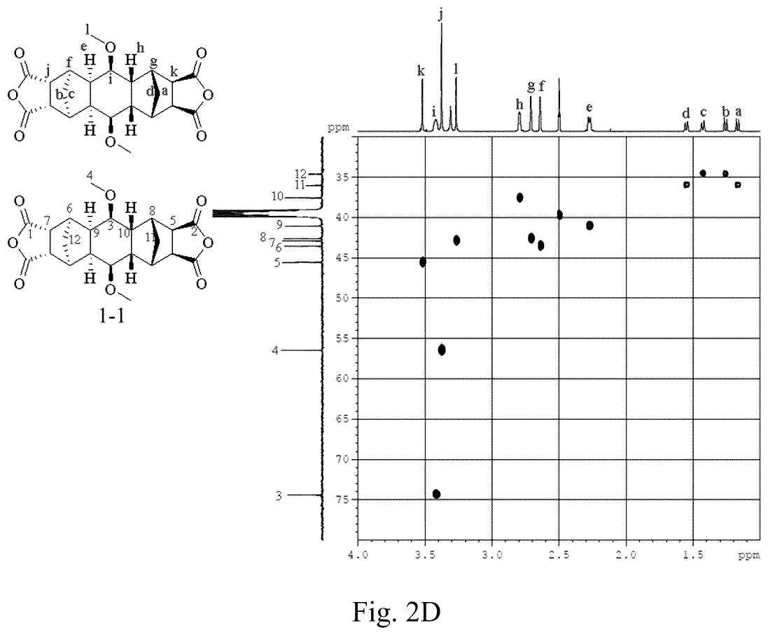

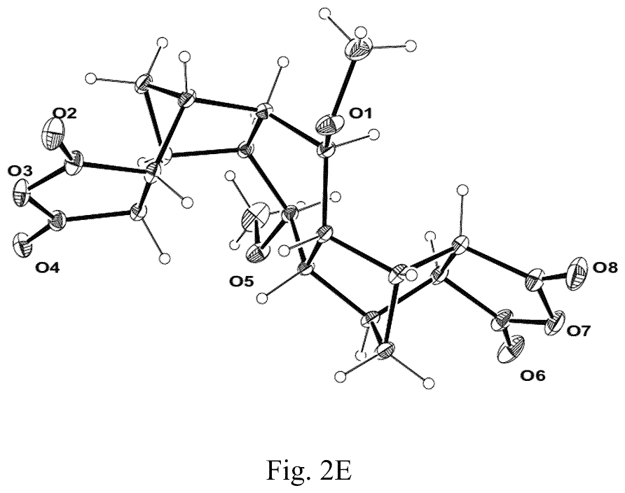

The ¹H NMR spectrum of compound 1-1 (cis-OMeDA) is shown in FIG. 2A, the ¹H¹H—COSY spectrum of compound 1-1 is shown in FIG. 2B, the ¹³C NMR spectrum of compound 1-1 is shown in FIG. 2C, the ¹³C¹H—HSQC spectrum of compound 1-1 is shown in FIG. 2D, and the X-ray single crystal diffraction pattern of compound 1-1 is shown in FIG. 2E.

The melting point of compound 1-1 (cis-OMeDA) is 304° C. The NMR hydrogen spectrum of compound 1-1 (cis-OMeDA) is “¹H NMR (600 MHz, DMSO) δ 3.52 (d, J=1.3 Hz, 2H), 3.42 (d, J=3.7 Hz, 2H), 3.38 (s, 6H), 3.27 (d, J=1.0 Hz, 2H), 2.80 (dd, J=4.1, 2.0 Hz, 2H), 2.71 (s, 2H), 2.64 (s, 2H), 2.32-2.24 (m, 2H), 1.55 (d, J=11.2 Hz, 1H), 1.43 (d, J=11.4 Hz, 1H), 1.26 (d, J=11.4 Hz, 1H), 1.17 (d, J=11.2 Hz, 1H).” The NMR carbon spectrum of compound 1-1 (cis-OMeDA) is “¹³C NMR (150 MHz, DMSO, 5, ppm): 174.54, 174.05, 74.40, 56.45, 45.57, 43.57, 42.91, 42.63, 41.08, 37.58, 36.03, 34.63.” The mass spectrum of compound 1-1 (cis-OMeDA) is “EIMS (m/z): calcd for C₂₂H₂₄O₈, 416.2; found 416.2 [M]⁺. Anal. Calcd for C₂₆H₃₆O₁₀: C, 63.45; H, 5.81; 0, 30.74; found, C, 63.41; H, 5.86; 0, 31.08.”

Synthesis of Compound 1a-2(Cis-OEt):

A 500 mL three-necked flask flushed with nitrogen and equipped with a condenser and magnetic stirrer was added with 5 g (20.5 mmol) of compound 1c (cis-OH) and 200 mL of tetrahydrofuran. A 50 mL sample bottle was prepared and 6.55 g (163.9 mmol) of 60% sodium hydride (NaH) was added, and the mineral oil was rinsed with n-hexane. After the solvent is pumped out with a water pump, it is slowly added into the three-necked flask in batches under an ice bath to form a white suspension solution. During the dosing process, it should be noted that sodium hydride would react with water in tetrahydrofuran to release heat and produce hydrogen, so it must be added slowly in batches. Then, 17.86 g (163.9 mmol) of ethyl bromide (CH₃CH₂Br) was added into the three-necked flask. Because ethyl bromide is volatile, it must be added directly to the solution to avoid an unbalanced dosage ratio. The mixture was then stirred at 60° C. under reflux for 2 days.

After the reaction, thin layer chromatography was performed, in which ethyl acetate (EA):n-hexane=1:5. The thin layer chromatography piece was then confirmed using potassium permanganate colorimetric solution. The Rt value of compound 1a-2 (cis-OEt) is 0.8, the Rt value of the byproduct that reacts only with the ethyl group on the upper side is 0.8, and the Rt value of the starting material is 0.8. After cooling, saturated aqueous ammonium chloride solution was added to terminate the reaction. The remaining sodium hydride in the three-necked flask was reacted until no bubbles escaped.

The tetrahydrofuran solution was then concentrated under reduced pressure. At this time, there would be a small amount of white solid starting compound 1c (cis-OH) and a yellow ointment mixture (compound 1a-2 (cis-OEt) and by-products) in the conical flask. The yellow ointment was dissolved with ether and then filtered by suction to recover the starting material. The lower filtrate was collected and extracted with ether and saturated brine. The organic layer was washed with saturated brine until neutral, and then dehydrated with anhydrous magnesium sulfate. The filtrate was collected by suction filtration. After the solvent is removed by concentration under reduced pressure, a pale yellow liquid crude product is obtained.

The yellow liquid crude product is then used with the above-mentioned proportion of solvent to prepare two short columns of thin layer chromatography sheet height for chromatography separation of by-products. After drying in a vacuum oven at room temperature, 4 g of compound 1a-2 (cis-OEt) was obtained with a yield of 65%, 1.39 g of by-products with a yield of 25%, and 0.5 g of starting material.

Synthesis of Compound 1b-2(Cis-OEtTE):

A 250 mL glass container was placed in a nitrogen-filled autoclave flushed with nitrogen, and 3 g (10 mmol) of compound 1a-2 (cis-OEt) was added, followed by 92 mL (2.2 mol) of anhydrous methanol (MeOH), 11.61 g (80.64 mmol) of copper (II) chloride (CuCl₂), 0.3032 g (0.285 mmol) of palladium on carbon (Pd/C), and 17.71 g (161.2 mol) of anhydrous sodium acetate. It should be noted that the catalyst must be dehydrated at 100° C. in vacuum for more than 6 hours before use. When adding the palladium carbon catalyst to the container, the stirring rate must be increased (350 rpm) and added slowly under nitrogen, otherwise there is a risk of fire when the palladium carbon catalyst contacts methanol under air. After dosing, the autoclave was locked and a carbon monoxide cylinder was connected. The pressure was adjusted to 2 kg/cm², and the pressure relief valve was slightly opened for 15 minutes to replace the pressure in the autoclave with carbon monoxide. Then the pressure was adjusted to 3 kg/cm² for half an hour and the autoclave was observed for leaks. Finally, the pressure was adjusted to 4.5 kg/cm² and stirred at 380 rpm for two days at room temperature.

After the reaction completed, the carbon monoxide in the autoclave was slowly discharged until the pressure returned to 1 atmosphere. 50 mL of chloroform was added into the container and stirred for 15 minutes, and then the catalyst was filtered through diatomaceous earth. The lower filtrate was taken and extracted with chloroform and saturated brine, and the organic layer was washed with saturated brine until neutral. During the process, the organic layer, which was originally blue-green, will turn into a light yellow. As the number of extractions increases, the water layer will change from blue-green to transparent and white mist. The organic layer was collected and anhydrous magnesium sulfate was added to remove water. The filtrate was collected by vacuum filtration and concentrated under reduced pressure to remove the solvent to obtain a pale yellow ointment liquid crude product.

The crude ointment was added with a little ether (about 5 mL) for recrystallization and placed in a refrigerator overnight. The upper crystals were collected by vacuum filtration. The lower filtrate was recovered, concentrated under reduced pressure to dry the solvent, and then the above recrystallization step was repeated. After drying in a vacuum oven at 70° C. for 12 hours, 2.07 g of white crystals of compound 1b-2 (cis-OEtTE) were obtained with a yield of 39%.

### Synthesis of Compound 1-2 (Cis-OEtDA):

The IUPAC of compound 1-2 is named as: (3aR,4R,5S,6R,6aR,9aS,10S,11R,12S,12aS)-5,11-diethoxytetradecahydro-1H, 3H-4,12:6,10-dimethanoanthra[2,3-c:6,7-c′]difuran-1,3,7,9-tetraone.

A 25 mL three-necked round-bottom flask flushed with nitrogen and equipped with a dosing tube, a condenser and a magnetic stirrer was added with 0.85 g (1.59 mmol) of compound 1b-2 (cis-OEtTE), 1.5 mL of formic acid, 1.5 mL of pure water and 0.3 mL of hydrochloric acid (37%) in sequence. The reaction temperature was adjusted to 90° C. and the mixture was completely dissolved after 4 hours of reaction. After the reaction was completed, white crystals will precipitate when the temperature was lowered to room temperature. In addition, the upper layer of crystals is retained by vacuum filtration and dried in a 100° C. high-temperature oven for one day to obtain white crystals. Then, 4 mL of acetic anhydride was slowly added by using a dosing tube to avoid sudden boiling. After the acetic anhydride was completely added, the temperature was adjusted to 120° C. and the reaction was carried out for 12 hours. After the reaction was completed, the dosing tube and reflux tube were removed. A vacuum distillation apparatus was set up to distill most of the solvent, which was then vacuum filtered and most of the acetic anhydride was washed with ether. After being dried in a high-temperature oven at 100° C. for half a day, 0.7 g of crude product compound 1-2 (cis-OEtDA) was obtained with a yield of 99.9%.

The crude product was recrystallized in a nitrogen atmosphere at a ratio of 1 mL of acetic anhydride to 1 g of the crude product overnight. The mixture was filtered under vacuum and rinsed with ether and the upper solid layer was collected. After drying in a high-temperature oven at 140° C. for one day, 0.42 g of compound 1-2 (cis-OEtDA) was obtained as white crystals with a yield of 60%. The crude product could also be sublimated to remove color and impurities, and pure compound 1-2 (cis-OEtDA) could be obtained as a white powder.

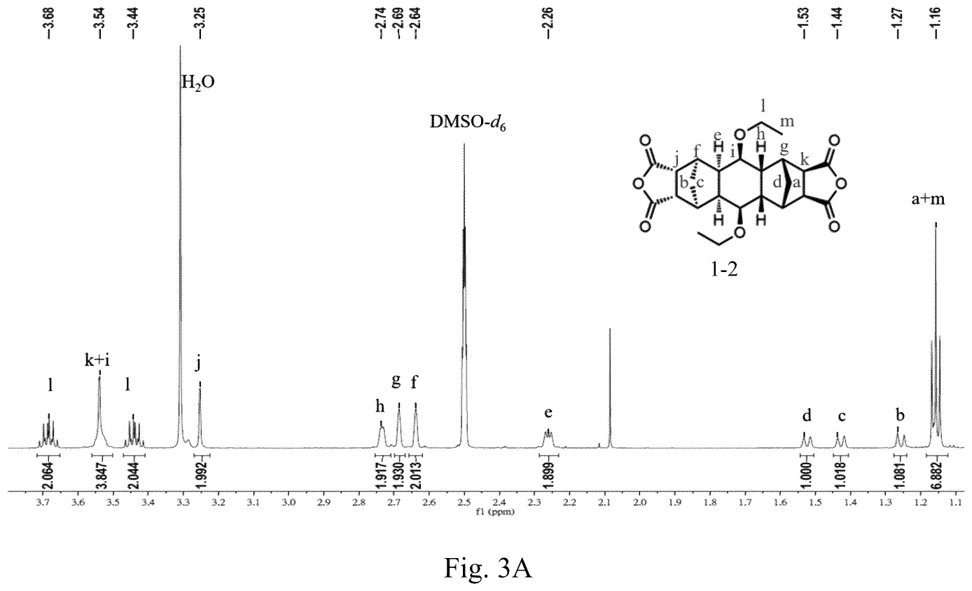

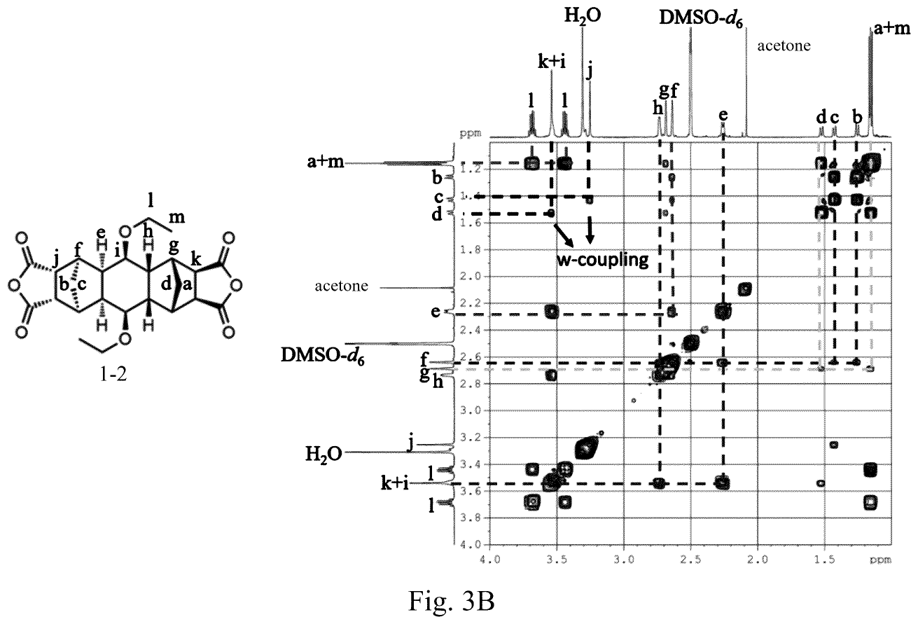

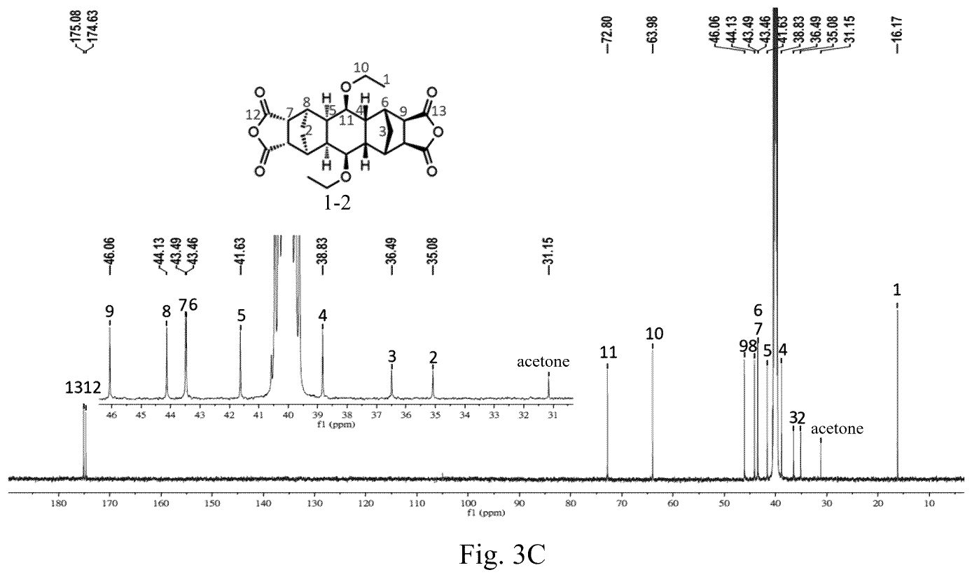

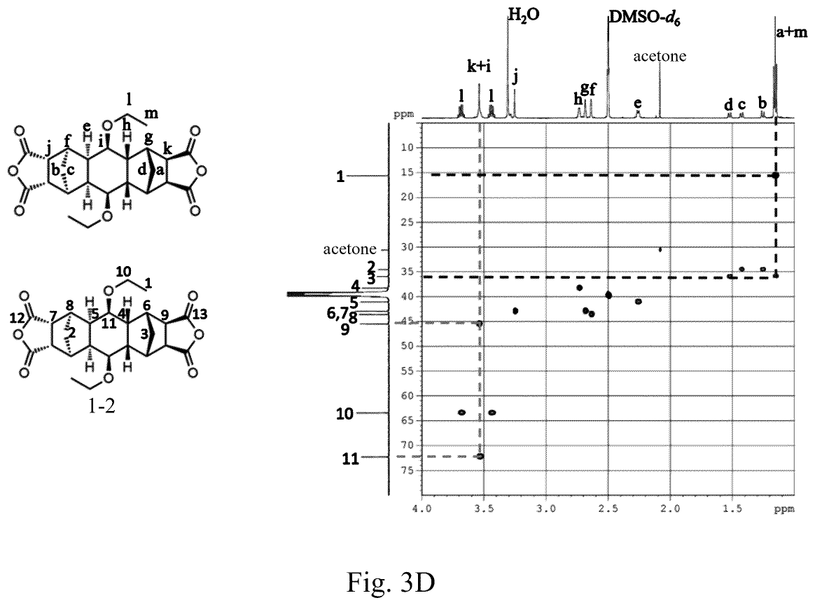

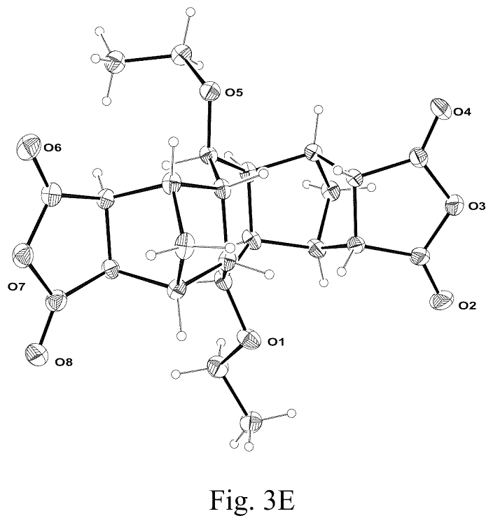

The ¹H NMR spectrum of compound 1-2 (cis-OEtDA) is shown in FIG. 3A, the ¹H¹H—COSY spectrum of compound 1-2 is shown in FIG. 3B, the ¹³C NMR spectrum of compound 1-2 is shown in FIG. 3C, the ¹³C¹H—HSQC spectrum of compound 1-2 is shown in FIG. 3D, and the X-ray single crystal diffraction pattern of compound 1-2 is shown in FIG. 3E.

The melting point of compound 1-2 (cis-OEtDA) is 254° C. The NMR hydrogen spectrum of compound 1-2 (cis-OEtDA) is “¹H NMR (600 MHz, DMSO-d₆, δ, ppm): 3.68 (dq, J=9.6, 7.0 Hz, 2H), 3.58 (m, 2H), 3.54 (d, J=1.3 Hz, 2H), 3.54 (m, 2H), 3.44 (dq, J=9.6, 7.0 Hz, 2H), 3.25 (d, J=1.1 Hz, 2H), 2.74 (m, 2H), 2.69 (s, 2H), 2.64 (s, 2H), 2.26 (m, 2H), 1.53 (d, J=11.1 Hz, 1H), 1.44 (d, J=11.4 Hz, 1H), 1.27 (d, J=11.3 Hz, 1H), 1.16 (t, J=7.0 Hz, 6H), 1.16 (m, 1H).” The NMR carbon spectrum of compound 1-2 (cis-OEtDA) is “¹³C NMR (150 MHz, DMSO-d₆, δ, ppm): 175.08, 174.63, 72.80, 63.98, 46.06, 44.13, 43.49, 43.46, 41.63, 38.83, 36.49, 35.08, 31.15, 16.17.” The mass spectrum of compound 1-2 (cis-OEtDA) is “EIMS (m/z): calcd for C₂₄H₂₈O₈⁺: 444; found 444 [M]⁺. Anal. calcd for C₂₄H₂₈O₈: C, 64.85; H, 6.35; O, 28.80; found, C, 64.25; H, 6.34; O, 29.29.”

### Synthesis of Compound 2a (Trans-OMe):

A 500 mL three-necked flask flushed with nitrogen and equipped with a condenser and magnetic stirrer was added with 2 g (8.2 mmol) of compound 2c (trans-OH) and 200 mL of tetrahydrofuran. A 50 mL sample bottle was prepared and 2.62 g (65.6 mmol) of 60% sodium hydride (NaH) was added, and the mineral oil was rinsed with n-hexane. After the solvent is pumped out with a water pump, it is slowly added into the three-necked flask in batches under an ice bath to form a white suspension solution. During the dosing process, it should be noted that sodium hydride would react with water in tetrahydrofuran to release heat and produce hydrogen, so it must be added slowly in batches. Then, 9.3 g (65.6 mmol) of methyl iodide (CH₃I) was added into the three-necked flask. Because methyl iodide is volatile, it must be added directly to the solution to avoid an unbalanced dosage ratio. Then the mixture was stirred at 60° C. in a closed reflux chamber for half a day.

Half a day later, the completion of the reaction was confirmed by thin layer chromatography, in which ethyl acetate (EA):n-hexane=1:5. A potassium permanganate colorimetric solution was used to confirm that there is only one spot on the thin layer chromatography sheet (i.e. Rt of compound 2a=0.5). After cooling, saturated aqueous ammonium chloride solution was added to terminate the reaction. The remaining sodium hydride in the three-necked flask was reacted until no bubbles escaped. The tetrahydrofuran solution was then concentrated under reduced pressure and extracted with diethyl ether and saturated brine. The organic layer was washed with saturated brine until neutral, and then dehydrated with anhydrous magnesium sulfate. The filtrate was collected by vacuum filtration and concentrated under reduced pressure to remove the solvent, thereby obtaining a light yellow liquid product compound 2a (trans-OMe). After drying in a vacuum oven at room temperature, 2.22 g of compound 2a (trans-OMe) was obtained with a yield of 99.9%.

### Synthesis of Compound 2b (Trans-OMeTE):

A 250 mL glass container was placed in a nitrogen-filled autoclave flushed with nitrogen, and 2 g (7.35 mmol) of compound 2a (trans-OMe) was added. Then, 67 mL (1.46 mol) of anhydrous methanol, 8.53 g (63.47 mmol) of copper (II) chloride, 0.223 g (0.209 mmol) of palladium carbon catalyst (Pd/C), and 13.01 g (126.94 mmol) of anhydrous sodium acetate were added in sequence. It should be noted that the catalyst must be dehydrated at 100° C. in vacuum for more than 6 hours before use. When adding the palladium carbon catalyst to the container, the stirring rate must be increased (350 rpm) and added slowly under nitrogen, otherwise there is a risk of fire when the palladium carbon catalyst contacts methanol under air. After dosing, the autoclave was locked and a carbon monoxide cylinder was connected. The pressure was adjusted to 2 kg/cm², and the pressure relief valve was slightly opened for 15 minutes to replace the pressure in the autoclave with carbon monoxide. Then the pressure was adjusted to 3 kg/cm² for half an hour and the autoclave was observed for leaks. Finally, the pressure was adjusted to 4.5 kg/cm² and stirred at 350 rpm for two days at room temperature.

After the reaction completed, the carbon monoxide in the autoclave was slowly discharged until the pressure returned to 1 atmosphere. 50 mL of chloroform was added into the container and stirred for 15 minutes, and then the catalyst was filtered through diatomaceous earth. The lower filtrate was taken and extracted with chloroform and saturated brine, and the organic layer was washed with saturated brine until neutral. During the process, the organic layer, which was originally blue-green, will turn into a light yellow. As the number of extractions increases, the water layer will change from blue-green to transparent and white mist. The organic layer was collected and anhydrous magnesium sulfate was added to remove water. The filtrate was collected by vacuum filtration and concentrated under reduced pressure to remove the solvent to obtain a 3.8 g of a crude light yellow ointment product (compound 2b (trans-OMeTE)). The product was dried in a vacuum oven at 50° C. for 12 hours without purification and directly used for the next step.

Synthesis of Compound 2b′ (Trans-OMeTA):

A 100 mL three-necked flask with a reflux tube was set up, and 3.8 g of the crude compound 2b (trans-OMeTE), 30 mL of methanol, and 30 mL of a 2M sodium hydroxide aqueous solution were added to the three-necked flask in sequence, and the mixture was refluxed and stirred for 4 hours. The solution was turbid at first, but would become clear and transparent after the reaction, with a small amount of salt precipitation.

After the reaction was completed and the temperature was lowered, the by-products from the previous autoclave in the water layer were extracted twice with ethyl acetate. The aqueous layer was retained and 12 M hydrogen chloride was slowly added under ice bath until the solution was neutral. The ice bath was removed and 12 M hydrogen chloride was added at room temperature until the solution was weakly acidic, for example, pH test paper appears pink. During the process, a white precipitate gradually precipitated out and the mixture was stirred at 50° C. for 15 minutes. Then, saturated brine was added and the mixture was extracted twice with a large amount of ethyl acetate (500 mL in total). The white precipitate would dissolve in the organic layer, and the color of the organic layer would turn light yellow. The organic layer did not need to be extracted to neutrality. The organic layer was directly collected and anhydrous magnesium sulfate was added to remove water. The filtrate was collected by vacuum filtration and concentrated under reduced pressure to remove the solvent to obtain a white solid crude product containing yellow ointment. The crude product was added with a little glacial ethyl acetate (about 10 mL) and shaken in a shaker for several minutes. Most of the ointment will dissolve in ethyl acetate. The upper solid was collected by suction filtration. 1.86 g of compound 2b′ (trans-OMeTA) was obtained as a white solid after drying in a vacuum oven at 70° C. After purification, the total yield was estimated to be 55%.

### Synthesis of Compound 2 (Trans-OMeDA):

The IUPAC of compound 2 is named as: (3aR,4R,4aR,5R,5aR,6R,6aR,9aS,10S,10aS,11S,11aS,12S,12aS)-5,11-dimethoxytetradecahydro-1H,3H-4,12:6,10-dimethanoanthra[2,3-c:6,7-c′]difuran-1,3,7, 9-tetraone.

A 25 mL three-necked flask fluxing with nitrogen, condenser tube and mechanical stirrer were installed for reaction. 3.72 g (6.24 mmol) of compound 2b′ (trans-OMeTA) was added into a three-necked flask, and 8 mL of acetic anhydride was slowly added dropwise using a dosing tube. It is noted that acetic anhydride was added too quickly would cause sudden boiling. The reaction mixture was then heated to 120° C. and refluxed with stirring for 12 hours.

After the reaction was completed, the dosing tube and reflux tube were removed. A vacuum distillation apparatus was set up to distill most of the acetic anhydride and leave a small amount of acetic anhydride (about 0.5 ml). After cooling, a lot of white solid would precipitate. If no solid precipitates after cooling, the temperature was raised again to distill more solvent or 1 or 2 drops of acetic acid (poor solvent) was added before cooling to precipitate solid crystals of acid anhydride.

If the precipitated product (1 g) was very dark in color, it could be recrystallized from a mixed solution of acetic acid and acetic anhydride (1 mL, ratio 1:1) under nitrogen. It is noted that excessive nitrogen flow would dry out the solvent. After leaving it for half a day, many crystals could be seen. It was filtered under vacuum and the acetic anhydride was washed out with ether. The upper crystals were collected, and finally 2.46 g of compound 2 (trans-OMeDA) crystals were obtained, with a recrystallization yield of 72%.

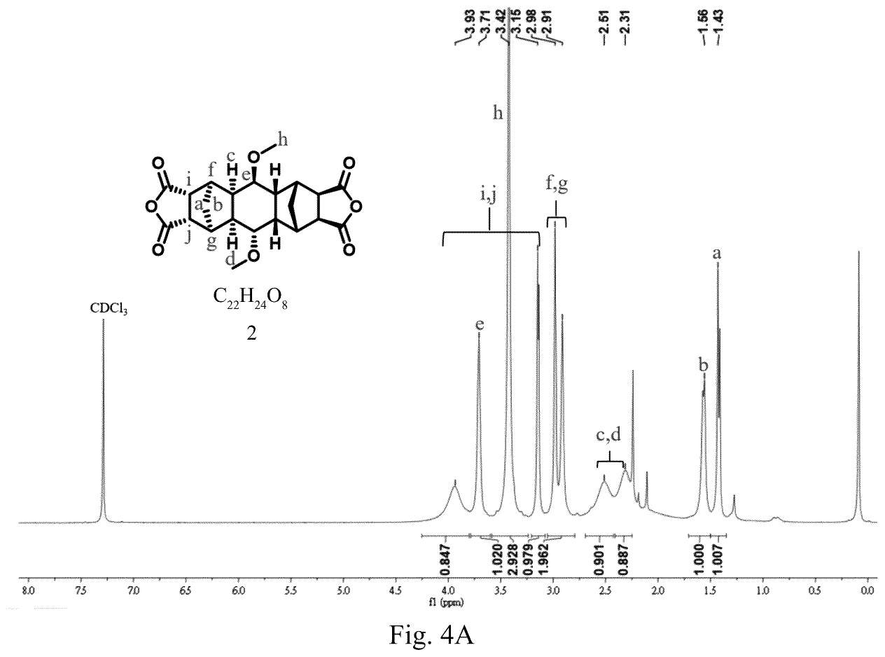

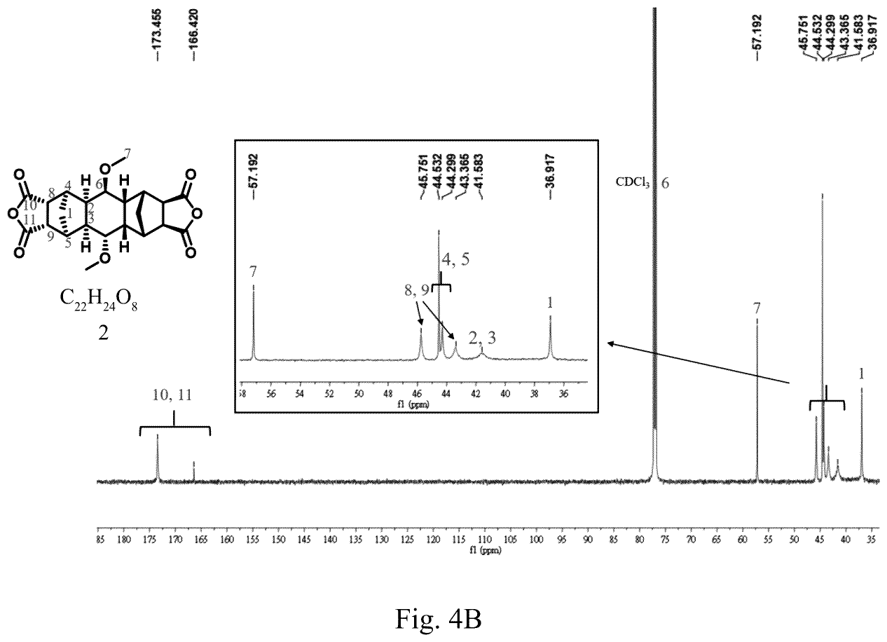

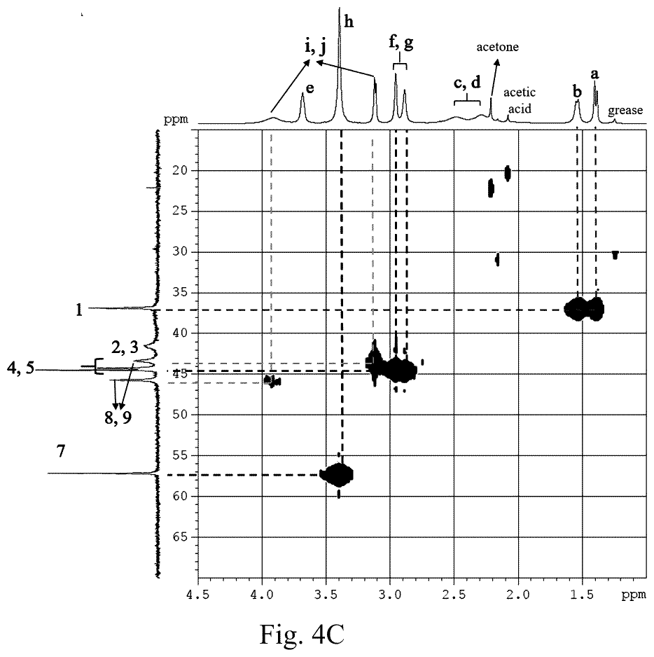

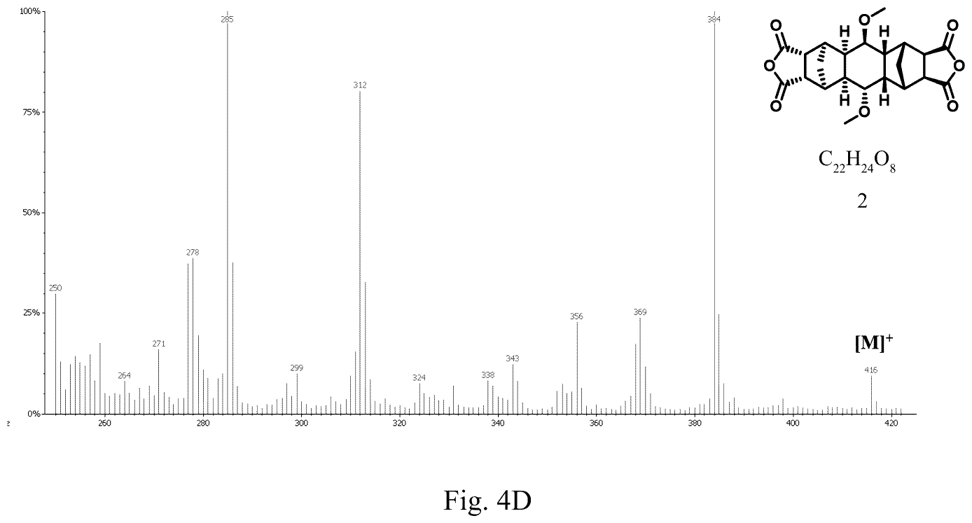

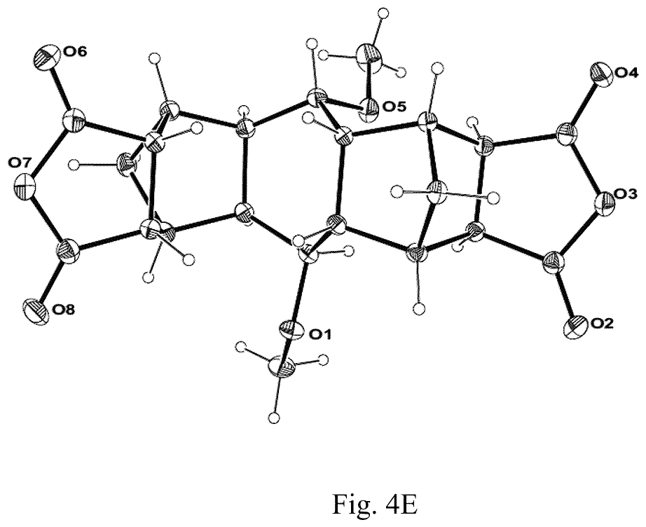

The ¹H NMR spectrum of compound 2 (trans-OMeDA) is shown in FIG. 4A, the ¹³C NMR spectrum of compound 2 is shown in FIG. 4B, the ¹³C¹H—HSQC spectrum of compound 2 is shown in FIG. 4C, the EI-MASS spectrum of compound 2 is shown in FIG. 4D, and the X-ray single crystal diffraction pattern of compound 2 is shown in FIG. 4E.

The melting point of compound 2 (trans-OMeDA) is 147° C. The NMR spectrum of compound 2 (trans-OMeDA) is “¹H NMR (600 MHz, DMSO-d₆, δ, ppm): δ3.93 (broad, 2H), 3.71 (s, 2H), 3.42 (s, 6H), 3.15 (m, 2H), 2.98 (s, 2H), 2.91 (s, 2H), 2.51 (broad, 2H), 2.31 (broad, 2H), 1.56 (broad, 2H), 1.43 (d, J=9.7 Hz, 2H).” The NMR carbon spectrum of compound 2 (trans-OMeDA) is “¹³C NMR (150 MHz, DMSO-d₆, δ, ppm): 173.45, 166.42, 57.19, 45.75, 44.53, 44.29, 43.36, 41.58, 41.58, 36.91.” The mass spectrum of compound 2 (trans-OMeDA) is “EI MS (m/z): calcd for C₂₂H²⁴O₈⁺: 416; found 416 [M]⁺. Anal. calcd for C₂₂H₂₄O₈: C, 63.45; H, 5.81; O, 30.74; found, C, 62.92; H, 5.77; O, 30.64.”

### Synthesis of Compound 3a (PhOMe):

A 500 mL three-necked round-bottom flask equipped with a magnetic stirrer was added with 7.9956 g (33.27 mmol) of compound 3c (BQCP) and 200 mL of tetrahydrofuran and dissolved at room temperature. After adding 4.5001 g (40.10 mmol) of potassium tert-butoxide and stirring for 2 hours, 12.5748 g (112.06 mmol) of potassium tert-butoxide was further added. After two hours of reaction, 19.2309 g (135.49 mmol) of methyl iodide was slowly added through the dosing tube at a rate of about 1 drop per second to react for 12 hours. After the reaction was completed, the mixture was extracted with ethyl acetate/saturated brine. After the organic layer was washed to neutrality, the organic layer was collected and anhydrous magnesium sulfate was added to remove moisture. The mixture was concentrated under reduced pressure and then dried in a vacuum oven at room temperature for 12 hours to obtain 5.4012 g (crude yield: 60.9%) of a brown solid crude product. A small amount of ethanol was added to the obtained brown solid crude product, and the mixture was vigorously stirred in an ice bath for about half an hour. The brown impurity will dissolve in ethanol, and the target product compound 3a (PhOMe) will precipitate. The white solid was collected by filtration and the solid surface was rinsed with a small amount of ice-cold ethanol. The product was dried in a vacuum oven at room temperature for 12 hours to obtain 4.2298 g of compound 3a (PhOMe) as a white solid with a yield of 47.7%.

### Synthesis of Compound 3b (PhOMeTE):

A 200 mL glass container was placed in a nitrogen-filled autoclave, and 2.66 g (10 mmol) of compound 3a (PhOMe) was added. Then, 92 mL (2.2 mol) of anhydrous methanol, 11.61 g (80.64 mmol) of copper (II) chloride, 0.3032 g (0.285 mmol) of palladium carbon catalyst (10%), and 17.71 g (161.2 mol) of anhydrous sodium acetate were added in sequence.

Before using 10% palladium carbon catalyst, water must be removed under vacuum at 100° C. for more than 6 hours to ensure anhydrous and then slowly added under nitrogen. After dosing, the autoclave was locked and a carbon monoxide cylinder was connected. The pressure was adjusted to 2 kg/cm², and the pressure relief valve was slightly opened for 15 minutes to replace the pressure in the autoclave with carbon monoxide. After the ventilation was completed, the pressure was adjusted to 5 kg/cm² and the reaction was vigorously stirred at 550 rpm at room temperature for two days.

### Synthesis of Compound 3 (PhOMeDA):

The IUPAC of compound 3 is named as: tetramethyl (1R,2S,3R,4S,5S,6R,7S,8R)-9,10-dimethoxy-1,2,3,4,5,6,7,8-octahydro-1,4:5,8-dimethanoanthracene-2,3,6,7-tetracarboxylate.

A 25 mL three-necked round-bottom flask was flushed with nitrogen and equipped with a dosing tube, a condenser and a magnetic stirrer, and 0.80 g (1.59 mmol) of compound 3b (PhOMeTE), 1.5 mL of formic acid, 1.5 mL of pure water and 0.3 mL of hydrochloric acid (37%) were added in sequence. After the reaction is completed, white crystals will precipitate when the temperature drops to room temperature. The upper layer of crystals will be retained by vacuum filtration. After drying in a high-temperature oven at 100° C. for one day, white crystals were obtained. 4 mL acetic anhydride was then slowly drop by using a dosing tube to avoid sudden boiling. After the acetic anhydride was completely added, the temperature was adjusted to 120° C. and the reaction was carried out for 12 hours. After the reaction was completed, the dosing tube and reflux tube were removed. A vacuum distillation apparatus was set up to distill most of the solvent, which was then vacuum filtered and most of the acetic anhydride was washed with ether. After being dried in a high-temperature oven at 100° C. for half a day, 0.35 g of crude product compound 3 (PhOMeDA) was obtained with a yield of 53.7%.

The crude product was recrystallized under nitrogen atmosphere with 1 mL of acetic anhydride to 1 g of the crude product. Let the crystals cool to room temperature overnight. The solid surface was washed with ether by suction filtration and the upper solid was collected. After drying in a high-temperature vacuum oven at 140° C. for one day, 0.279 g of compound 3 (PhOMeDA) was obtained as white crystals with a yield of 42.8%.

### Synthesis of Polyimide Polymer:

A 25 mL three-necked flask fluxing with nitrogen, condenser tube and mechanical stirrer were installed for reaction. All monomers, solvents and catalysts were dried in a vacuum oven or purified by reduced pressure distillation to remove water. All diamines and dianhydrides used in the synthesis of polyimide polymers are synthesized in a 1:1 molar ratio.

The polyimide polymer is synthesized from the alicyclic dianhydride monomers containing ether group of Compound 1-1, Compound 1-2 and Compound 2 obtained in the present disclosure. The dianhydride monomer can be synthesized using any aromatic diamine or aliphatic diamine, for example, 4,4′-oxydianiline (ODA), 2,2′-Bis(trifluoromethyl)benzidine (TFMB), 9,9-Bis(4-aminophenyl)fluorene (FDA), and p-Phenylenediamine (PPD). According to the above synthesis steps, a polyimide polymer derived from the alicyclic dianhydride containing ether group was synthesized, and the results are shown in Table 1 below through gel permeation chromatography (GPC). The viscosity (η) in Table 1 is the intrinsic viscosity of the sample measured at 35° C. when the concentration is 0.5 g/dL in N-methylpyrrolidone (NMP); Mw is the weight average molecular weight obtained by GPC in N,N-dimethylacetamide (DMAc) (relative to polystyrene standards); Mn is the number average molecular weight obtained by GPC in N,N-dimethylacetamide (DMAc) relative to polystyrene standards; and PDI is the polydispersity index and is calculated as Mw/Mn.

TABLE 1

| Polyimide polymer | | | | |
|---|---|---|---|---|
| derived from alicyclic | | | | |
| dianhydride containing | η | Mw | Mn | |
| ether group | (dL/g) | (kDa) | (kDa) | PDI |
| Embodiment 1 | 1.03 | 471 | 186 | 2.53 |
| PI-cis OMeDA/ODA | | | | |
| Embodiment 2 | 0.95 | 83 | 28 | 2.93 |
| PI-cis OMeDA/TFMB | | | | |
| Embodiment 3 | 0.73 | 149 | 74 | 2.01 |
| PI-cis OMeDA/FDA | | | | |
| Embodiment 4 | 1.19 | 173 | 95 | 1.82 |
| PI-cis OEtDA/ODA | | | | |
| Embodiment 5 | 1.31 | 138 | 85 | 1.62 |
| PI-cis OEtDA/TFMB | | | | |
| Embodiment 6 | 1.09 | 154 | 107 | 1.43 |
| PI-cis OEtDA/FDA | | | | |
| Embodiment 7 | 1.04 | 120 | 100 | 1.20 |
| PI-trans OEtDA/ODA | | | | |
| Embodiment 8 | 1.11 | 72 | 58 | 1.23 |
| PI-trans OEtDA/TFMB | | | | |
| Embodiment 9 | 0.92 | 70 | 58 | 1.21 |
| PI-trans OEtDA/FDA | | | | |
| Embodiment 10 | 0.90 | 38 | 30 | 1.26 |
| PI-trans OEtDA/PPD | | | | |

Next, the solubility test results of the polyimide polymer derived from the alicyclic dianhydride containing ether group are shown in Table 2 below. Specifically, 20 mg of each sample was added to 1 mL solvent and stirred to test the solubility. The symbol “++” means to dissolve at room temperature, “+” means to dissolve when heated, and means not to dissolve when heated.

TABLE 2

| Polyimide polymer derived | | | | | | | | | |
|---|---|---|---|---|---|---|---|---|---|
| from alicyclic dianhydride | Solvents | | | | | | | | |
| containing ether group | m-Cresol | GBL | NMP | DMAC | DMSO | THF | Chloroform | Cyclopentanone | acetone |
| Embodiment 1 | ++ | ++ | ++ | ++ | ++ | − | + | + | − |
| PI-cis OMeDA/ODA | | | | | | | | | |
| Embodiment 2 | ++ | ++ | ++ | ++ | ++ | ++ | ++ | ++ | ++ |
| PI-cis OMeDA/TFMB | | | | | | | | | |
| Embodiment 3 | ++ | ++ | ++ | ++ | ++ | + | ++ | ++ | − |
| PI-cis OMeDA/FDA | | | | | | | | | |
| Embodiment 4 | ++ | ++ | ++ | ++ | ++ | − | ++ | + | − |
| PI-cis OEtDA/ODA | | | | | | | | | |
| Embodiment 5 | ++ | ++ | ++ | ++ | ++ | ++ | ++ | ++ | ++ |
| PI-cis OEtDA/TFMB | | | | | | | | | |
| Embodiment 6 | ++ | ++ | ++ | ++ | ++ | ++ | ++ | ++ | − |
| PI-cis OEtDA/FDA | | | | | | | | | |
| Embodiment 7 | ++ | ++ | ++ | ++ | ++ | − | ++ | + | − |
| PI-trans OEtDA/ODA | | | | | | | | | |
| Embodiment 8 | ++ | ++ | ++ | ++ | ++ | ++ | ++ | ++ | ++ |
| PI-trans OEtDA/TFMB | | | | | | | | | |
| Embodiment 9 | ++ | ++ | ++ | ++ | ++ | + | ++ | ++ | − |
| PI-trans OEtDA/FDA | | | | | | | | | |

The thermal stability of the Polyimide polymer derived from alicyclic dianhydride containing ether group was tested below, and the results are shown in Table 3 below, of which coefficient of thermal expansion (GTE) was measured in the range of 50° C. to 150° C., and glass transition temperature (T_{g}) was tested by thermal mechanical analyzer (TMA).

TABLE 3

| Polyimide polymer | | | Decomposition | Decomposition |
|---|---|---|---|---|
| derived from alicyclic | | | Temperature | Temperature |
| dianhydride containing | CTE | T_{g} | T_{d}^{5%} | T_{d}^{10%} |
| ether group | (ppm/K) | (° C.) | (° C.) | (° C.) |
| Embodiment 1 | 39.7 | 400 | 421 | 439 |
| PI-cis OMeDA/ODA | | | | |
| Embodiment 2 | 21.6 | 405 | 413 | 438 |
| PI-cis OMeDA/TFMB | | | | |
| Embodiment 3 | 42.3 | 427 | 432 | 455 |
| PI-cis OMeDA/FDA | | | | |
| Embodiment 4 | 48.4 | 374 | 414 | 432 |
| PI-cis OEtDA/ODA | | | | |
| Embodiment 5 | 29.4 | 376 | 397 | 420 |
| PI-cis OEtDA/TFMB | | | | |
| Embodiment 6 | 62.6 | 401 | 386 | 417 |
| PI-cis OEtDA/FDA | | | | |
| Embodiment 7 | 45.5 | 358 | 402 | 426 |
| PI-trans OEtDA/ODA | | | | |
| Embodiment 8 | 29.8 | 365 | 411 | 429 |
| PI-trans OEtDA/TFMB | | | | |
| Embodiment 9 | 52.1 | 393 | 413 | 437 |
| PI-trans OEtDA/FDA | | | | |
| Embodiment 10 | 39.7 | 368 | 410 | 429 |
| PI-trans OEtDA/PPD | | | | |
| Embodiment 11 | — | — | 415 | 431 |
| PI-trans OMeDA/PPD | | | | |
| Embodiment 12 | — | — | 387 | 426 |
| PI-trans OMeDA/BBH | | | | |

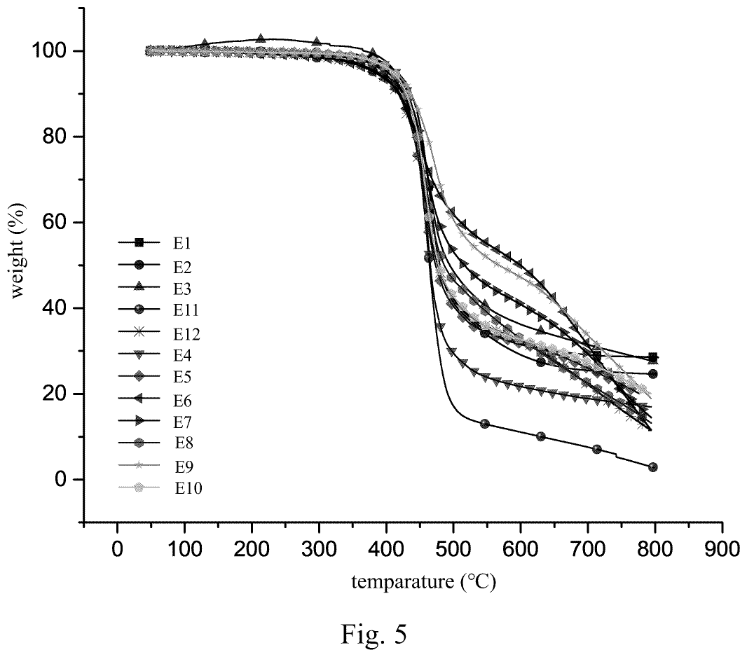

The Thermogravimetric analysis (TGA) results of the polyimide polymer derived from the alicyclic dianhydride containing ether group of Embodiment 1 to Embodiment 12 of the present disclosure are shown in FIG. 5.

Synthesis of Polyimide Films Derived from Alicyclic Dianhydride Containing Ether Group:

The polyimide polymer is dissolved in N, N-dimethylacetamide (DMAc) at a solid content of about 4% (w/v). After filtering using a 0.45 μm Teflon filter, the solution was poured onto a culture dish treated with isopropanol. After degassing, bake in a circulation oven at 60° C., 6 hours→160° C., 3 hours→100° C., 0.5 hours→60° C., 0.5 hours. The film is gently removed from the culture dish. The film is then dried in a vacuum oven at 200° C. to ensure that the solvent is completely removed, thereby obtaining a transparent polyimide polymer film with a film thickness of about 10 μm to 30 μm.

The various embodiments of the present disclosure are based on the above-mentioned method for synthesizing thin films to form polyimide films derived from the alicyclic dianhydride containing ether group. The optical property test results of the polyimide films derived from the alicyclic dianhydride containing ether group according to various embodiments of the present disclosure are listed in Table 4 below. λ_{80%} represents the wavelength at which the light transmittance of the film is 80%, λ_{cut-off} represents the wavelength at which the light transmittance of the film is less than 1%, and T₄₀₀(%) represents the light transmittance at the wavelength of 400 nm.

TABLE 4

| Polyimide film | | | | |
|---|---|---|---|---|
| derived from | | | | |
| alicyclic | | | | |
| dianhydride | | | | |
| containing ether | thickness | λ_{80%} | λ_{cut-off} | T₄₀₀ |
| group | (μm) | (nm) | (nm) | (%) |
| Embodiment 1 | 12 | 369 | 282 | 83.7 |
| PI-cis OMeDA/ODA | | | | |
| Embodiment 2 | 11 | 345 | 290 | 87.8 |
| PI-cis | | | | |
| OMeDA/TFMB | | | | |
| Embodiment 3 | 11 | 351 | 308 | 84.6 |
| PI-cis OMeDA/FDA | | | | |
| Embodiment 4 | 10 | 376 | 289 | 83.2 |
| PI-cis OEtDA/ODA | | | | |
| Embodiment 5 | 10 | 339 | 290 | 88.8 |
| PI-cis | | | | |
| OEtDA/TFMB | | | | |
| Embodiment 6 | 10 | 343 | 307 | 87.2 |
| PI-cis OEtDA/FDA | | | | |
| Embodiment 7 | 13 | 337 | 290 | 84.6 |
| PI-trans | | | | |
| OEtDA/ODA | | | | |
| Embodiment 8 | 10 | 374 | 285 | 83.2 |
| PI-trans | | | | |
| OEtDA/TFMB | | | | |
| Embodiment 9 | 11 | 424 | 313 | 76.1 |
| PI-trans | | | | |
| OEtDA/FDA | | | | |
| Embodiment 10 | 11 | 373 | 275 | 83.2 |
| PI-trans | | | | |
| OEtDA/PPD | | | | |

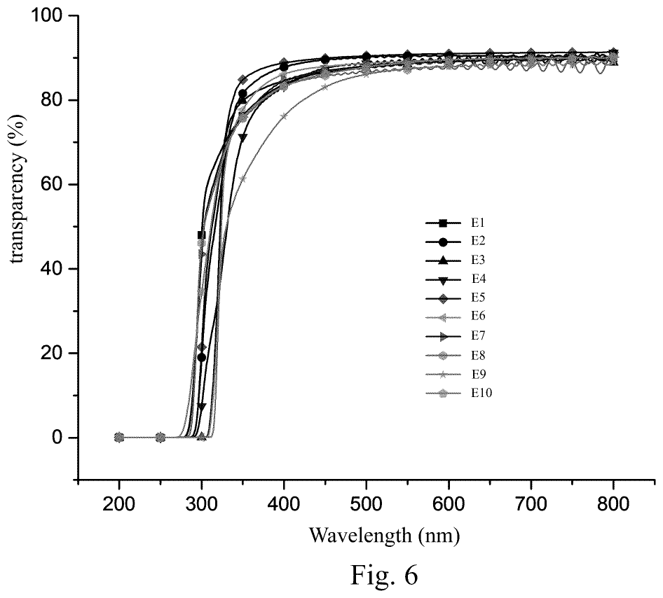

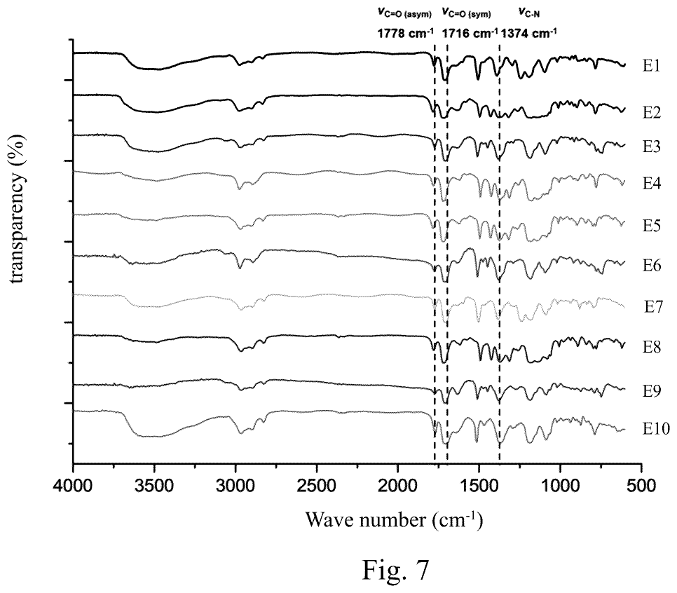

The UV-visible spectrum and the Fourier transform infrared spectrum of the polyimide film derived from the alicyclic dianhydride containing ether group of Embodiment 1 to Embodiment 10 of the present disclosure are shown in FIG. 6 and FIG. 7, respectively.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An alicyclic dianhydride containing ether group represented by a structure of the following formula (I), formula (II), or formula (III): 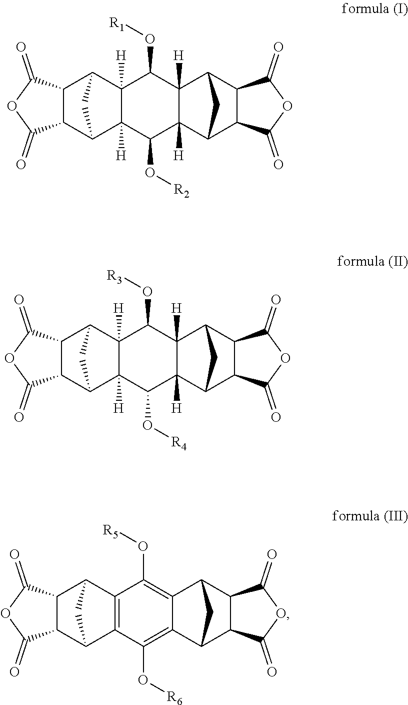
wherein R₁, R₂, R₃, R₄, R₅, and R₆ independently comprise a C1 to C5 hydrocarbon group.

2. The alicyclic dianhydride containing ether group of claim 1, wherein the alicyclic dianhydride containing ether group is represented by

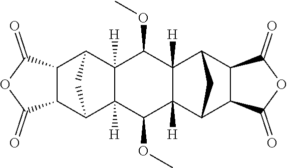 [SMILES: CO[C@H]1[C@@H]2[C@H]3C[C@H]([C@@H]4C(=O)OC(=O)[C@@H]43)[C@@H]2[C@@H](OC)[C@H]2[C@@H]3C[C@@H]([C@H]4C(=O)OC(=O)[C@H]43)[C@@H]12]

3. The alicyclic dianhydride containing ether group of claim 1, wherein the alicyclic dianhydride containing ether group is represented by

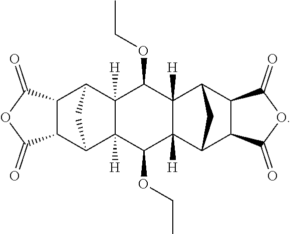 [SMILES: CCO[C@H]1[C@@H]2[C@H]3C[C@H]([C@@H]4C(=O)OC(=O)[C@@H]43)[C@@H]2[C@@H](OCC)[C@H]2[C@@H]3C[C@@H]([C@H]4C(=O)OC(=O)[C@H]43)[C@@H]12]

4. The alicyclic dianhydride containing ether group of claim 1, wherein the alicyclic dianhydride containing ether group is represented by

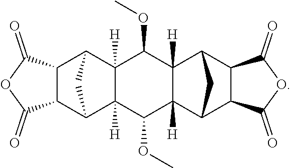 [SMILES: CO[C@H]1[C@@H]2[C@H]3C[C@H]([C@@H]4C(=O)OC(=O)[C@@H]43)[C@@H]2[C@H](OC)[C@H]2[C@@H]3C[C@@H]([C@H]4C(=O)OC(=O)[C@H]43)[C@@H]12]

5. The alicyclic dianhydride containing ether group of claim 1, wherein the alicyclic dianhydride containing ether group is represented by

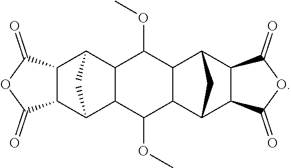 [SMILES: COC1C2C(C(OC)C3C1[C@H]1C[C@@H]3[C@@H]3C(=O)OC(=O)[C@@H]31)[C@@H]1C[C@H]2[C@H]2C(=O)OC(=O)[C@H]21]

6. A synthesis method of an alicyclic dianhydride containing ether group comprising:
performing a methoxy carbonylation reaction of a compound 1a to obtain a compound 1b, and performing a hydrolysis and dehydration reaction of the compound 1b to obtain the alicyclic dianhydride containing ether group represented by a formula (I), 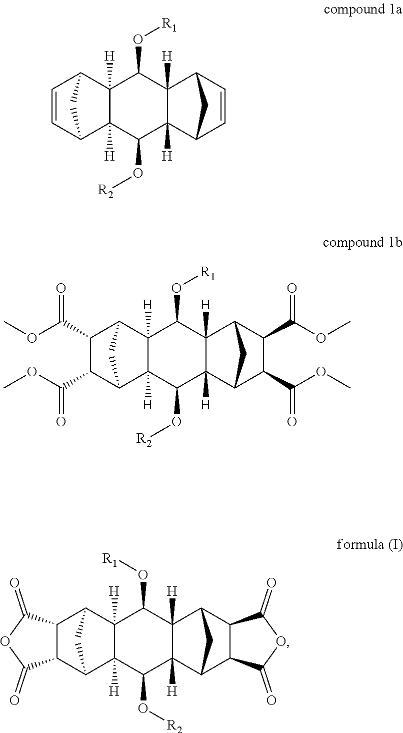
wherein R₁, R₂ independently comprise a C1 to C5 hydrocarbon group; or
performing a methoxy carbonylation reaction of a compound 2a to obtain a compound 2b, and performing a hydrolysis reaction and a dehydration reaction of the compound 2b to obtain the alicyclic dianhydride containing ether group represented by a formula (II), 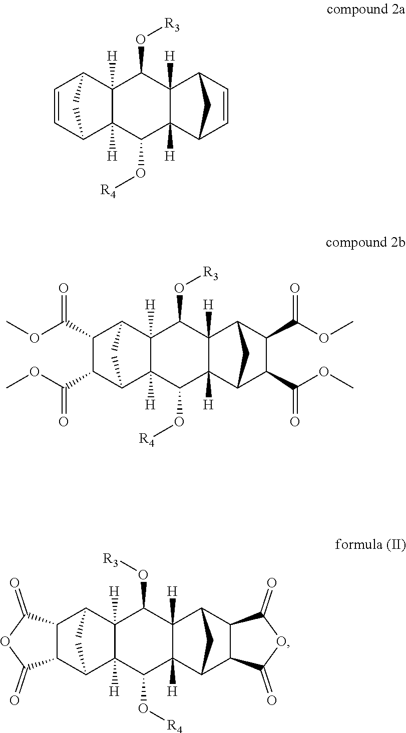
wherein R₃, R₄ independently comprise a C1 to C5 hydrocarbon group; or
performing a methoxy carbonylation reaction of a compound 3a to obtain a compound 3b, and performing a hydrolysis and dehydration reaction of the compound 3b to obtain the alicyclic dianhydride containing ether group represented by a formula (III), 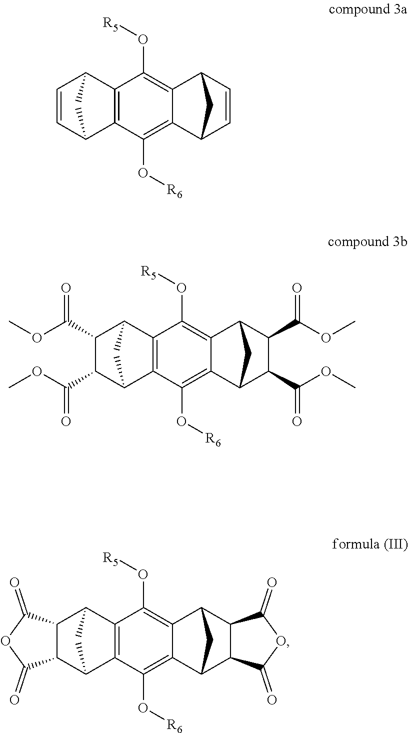
wherein R₅, R₆ independently comprise a C1 to C5 hydrocarbon group.

7. The synthesis method of the alicyclic dianhydride containing ether group of claim 6, wherein the methoxy carbonylation reaction comprises adding a palladium carbon catalyst.

8. The synthesis method of the alicyclic dianhydride containing ether group of claim 6, wherein the compound 1a is obtained by performing a Williamson ether synthesis reaction of a compound 1c, wherein the Williamson ether synthesis reaction comprises adding a haloalkane, and the haloalkane comprises a C1 to C5 haloalkane, and the compound 1c is represented by a structure as shown below:

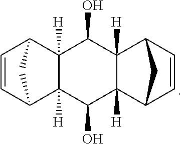 [SMILES: O[C@H]1[C@@H]2[C@@H]3C=C[C@@H](C3)[C@@H]2[C@@H](O)[C@H]2[C@H]3C=C[C@H](C3)[C@@H]12]

9. The synthesis method of the alicyclic dianhydride containing ether group of claim 6, wherein the compound 2a is obtained by performing a Williamson ether synthesis reaction of a compound 2c, wherein the Williamson ether synthesis reaction comprises adding a haloalkane, and wherein the haloalkane comprises a C1 to C5 haloalkane, and the compound 2c is represented by a structure as shown below:

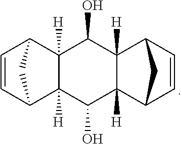 [SMILES: O[C@H]1[C@@H]2[C@@H]3C=C[C@@H](C3)[C@@H]2[C@H](O)[C@H]2[C@H]3C=C[C@H](C3)[C@@H]12]

10. The synthesis method of the alicyclic dianhydride containing ether group of claim 9, wherein the compound 2c is obtained by a reduction reaction of a compound 3c, wherein the reduction reaction comprises adding a lithium aluminium hydride, and the compound 3c is represented by a structure as shown below:

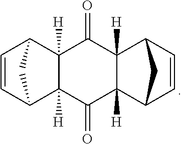 [SMILES: O=C1[C@@H]2[C@H](C(=O)[C@H]3[C@@H]1[C@H]1C=C[C@@H]3C1)[C@@H]1C=C[C@H]2C1]

11. The synthesis method of the alicyclic dianhydride containing ether group of claim 6, wherein the alicyclic dianhydride containing ether group represented by a formula (II) is obtained by performing the dehydration reaction of a compound 2b′, wherein the dehydration reaction comprises adding a acetic anhydride, and the compound 2b′ is represented by a structure as shown below:

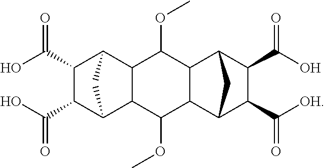 [SMILES: COC1C2C(C(OC)C3C1[C@H]1C[C@@H]3[C@H](C(=O)O)[C@@H]1C(=O)O)[C@@H]1C[C@H]2[C@@H](C(=O)O)[C@H]1C(=O)O]

12. The synthesis method of the alicyclic dianhydride containing ether group of claim 11, wherein the compound 2b′ is obtained by performing the hydrolysis reaction of the compound 2b, wherein the hydrolysis reaction comprises adding a methanol and a sodium hydroxide aqueous solution.

13. The synthesis method of the alicyclic dianhydride containing ether group of claim 6, wherein the compound 3a is obtained by performing an alkylation reaction of a compound 3c, wherein the alkylation reaction comprises adding a haloalkane, and the haloalkane comprises a C1 to C5 haloalkane, and the compound 3c is represented by a structure as shown below:

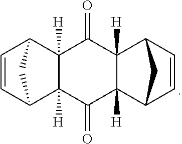 [SMILES: O=C1[C@@H]2[C@H](C(=O)[C@H]3[C@@H]1[C@H]1C=C[C@@H]3C1)[C@@H]1C=C[C@H]2C1]

14. A polyimide represented by a structure of the following formula (IV), formula (VI), or formula (VIII): 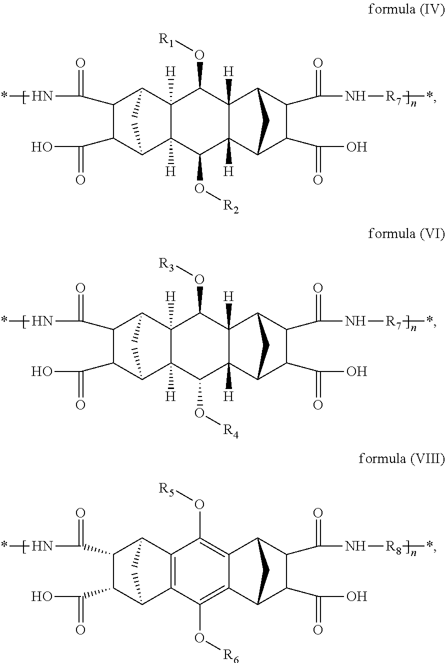
wherein R₁, R₂, R₃, R₄, R₅, and R₆ independently comprise a C1 to C5 hydrocarbon group, R₇ and R₈ independently comprise an aromatic group, an aliphatic group, a combination of an aromatic group and 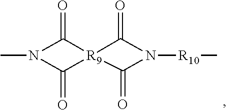
or a combination of aliphatic group and 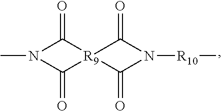
wherein R₉ is a moiety independently selected from the group consisting of substituted or unsubstituted aromatic, heterocyclic aromatic and aliphatic groups, Rio comprises an aromatic group or an aliphatic group, and n is a positive integer of 1 to 500.

15. A polyimide represented by a structure of the following formula (V), formula (VII), or formula (IX): 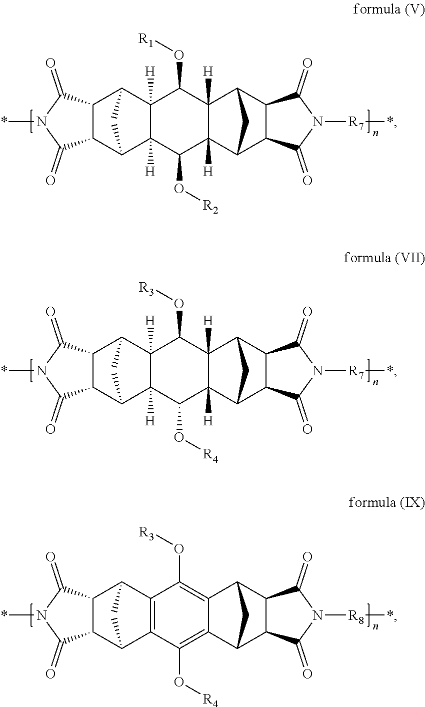
wherein R₁, R₂, R₃, R₄, R₅, and R₆ independently comprise a C1 to C5 hydrocarbon group, R₇ and R₈ independently comprise an aromatic group, an aliphatic group, a combination of an aromatic group and 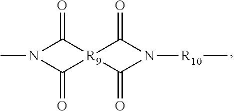
or a combination of aliphatic group and 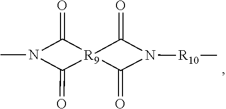
wherein R₉ is a moiety independently selected from the group consisting of substituted or unsubstituted aromatic, heterocyclic aromatic, and aliphatic groups, Rio comprises an aromatic group or an aliphatic group, and n is a positive integer of 1 to 500.
